(19) United States
(12) Patent Application Publication
(10) Pub. No.: US 2012/0108636 A1
(43) Pub. Date: May 3, 2012

(54) Allosteric Enhancers of th A1 Adenosine Receptor

(75) Inventors: Pier Giovanni Baraldi, Ferrara (IT); Allan R. Moorman, Durham, NC (US); Romeo Romagnoli, Ferrara (IT)

(73) Assignee: King Pharmaceuticals Research and Development, Inc.

(21) Appl. No.: 13/347,999

(22) Filed: Jan. 11, 2012

Related U.S. Application Data
(63) Continuation of application No. 12/437,344, filed on May 7, 2009, now abandoned.
(60) Provisional application No. 61/053,793, filed on May 16, 2008.
(60) Provisional application No. 61/051,399, filed on May 8, 2008.

(51) Int. Cl. A61K 31/4436; C07D 409/04; A61K 31/381; A61P 29/00; A61P 9/10; A61P 25/08; A61P 25/24; A61P 9/00; A61P 9/06; C07D 333/36; A61P 25/00

(52) U.S. Cl. 514/336; 549/68; 549/60; 549/59; 546/281.4; 514/447; 514/444

(57) ABSTRACT

The present invention provides compounds of formula (I)

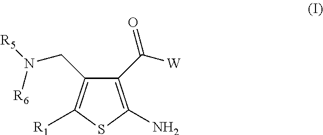

wherein W, R₁, R₅ and R₆ have a meaning as defined herein in the specification. The compounds of formula (I) are allosteric enhancers of the A₁ adenosine receptor and, thus, may be employed for the treatment of conditions mediated by the A₁ adenosine receptor. Accordingly, the compounds of formula (I) may be employed for treatment of pain, in particular, chronic pain such as neuropathic pain, and inflammatory pain, cardiac disease or disorder such as cardiac disarrhythmias, e.g., peroxysmal supraventricular tachycardia, angina, myocardial infarction and stroke, neurological disease or injury, sleep disorders, epilepsy and depression.

This application claims the benefit of U.S. Provisional Application No. 61/051,399 filed May 8, 2008 and U.S. Provisional Application No. 61/053,793 filed May 16, 2008, the entire contents of which are incorporated herein by reference.

The present invention relates to 2-aminothiophene derivatives, pharmaceutical compositions containing them, and to methods of treating conditions mediated by the A₁ adenosine receptor including pain, in particular, chronic pain such as neuropathic pain, and inflammatory pain, cardiac disease or disorder such as cardiac disarrhythmias, e.g., peroxysmal supraventricular tachycardia, angina, myocardial infarction and stroke, neurological disease or injury, sleep disorders, epilepsy and depression, by employing such compounds.

Accordingly, the present invention provides compounds of formula (I)

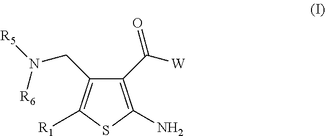

wherein
W is aryl, substituted aryl, heteroaryl, or substituted heteroaryl;
R₁ is hydrogen, alkyl, substituted alkyl, cycloalkyl, substituted cycloalkyl, alkenyl, substituted alkenyl, alkynyl, substituted alkynyl, aryl, substituted aryl, heteroaryl, or substituted heteroaryl;
R₅ and R₆ are, independently from each other, hydrogen, alkyl, substituted alkyl, aryl, substituted aryl, cycloalkyl, substituted cycloalkyl, heterocyclyl, or substituted heterocyclyl;
   or a pharmaceutically acceptable salt thereof.

The compounds of the present invention provide pharmacological agents which are allosteric enhancers of the A₁ adenosine receptor and, thus, may be employed for the treatment of conditions mediated by the A₁ adenosine receptor. Accordingly, the compounds of formula (I) may be employed for the treatment of pain, in particular, chronic pain such as neuropathic pain, and inflammatory pain, cardiac disease or disorder such as cardiac disarrhythmias, e.g., peroxysmal supraventricular tachycardia, angina, myocardial infarction and stroke, neurological disease or injury, sleep disorders, epilepsy and depression.

Listed below are definitions of various terms used to describe the compounds of the present invention. These definitions apply to the terms as they are used throughout the specification unless they are otherwise limited in specific instances either individually or as part of a larger group, e.g., wherein an attachment point of a certain group is limited to a specific atom within that group, the point of attachment is defined by an arrow at the specific atom.

The term “allosteric enhancer of the A₁ adenosine receptor” as used herein refers to a class of compounds that appear to enhance adenosine A₁ receptor function by stabilizing the high affinity state of the receptor-G-protein complex. This property may be measured as an increase in radioligand binding of an agonist to the adenosine A₁ receptor. An enhancer that increases agonist binding can do so by either accelerating the association of the agonist to the receptor, or by retarding the dissociation of the “receptor-ligand” complex and, therefore, must bind to a site different from the agonist recognition site. This putative site is termed the allosteric site, and presumably, compounds that bind to this site and enhance the agonist effect are termed as “allosteric enhancers”.

The term “alkyl” refers to a hydrocarbon chain having 1-20 carbon atoms, preferably 1-10 carbon atoms, and more preferably 1-7 carbon atoms. The hydrocarbon chain may be straight, as for a hexyl or n-butyl chain, or branched, as for example t-butyl, 2-methyl-pentyl, 3-propyl-heptyl. Exemplary alkyl groups include methyl, ethyl, propyl, isopropyl, n-butyl, t-butyl, isobutyl, pentyl, hexyl, isohexyl, heptyl, 4,4-dimethylpentyl, octyl, and the like.

The term “substituted alkyl” refers to those alkyl groups as described above substituted by one or more, preferably 1-3, of the following groups: halo, hydroxy, alkoxy, cycloalkyl, cycloalkoxy, alkylthio, alkylthiono, sulfonyl, sulfamoyl, carbamoyl, cyano, aryl, aryloxy, alkenyl, alkynyl, aralkoxy, optionally substituted amino, heterocyclyl including imidazolyl, furyl, thienyl, piperidinyl, pyrrolidyl, pyridyl, pyrimidyl, and the like.

The term “lower alkyl” refers to those alkyl groups as described above having 1-6, preferably 1-4 carbon atoms.

The term “alkenyl” refers to any of the above alkyl groups having at least two carbon atoms and further containing a carbon-to-carbon double bond at the point of attachment. Groups having 2-6 carbon atoms are preferred.

The term “alkynyl” refers to any of the above alkyl groups having at least two carbon atoms and further containing a carbon-to-carbon triple bond at the point of attachment. Groups having 2-6 carbon atoms are preferred.

The term “alkylene” refers to a straight-chain bridge of 2-5 carbon atoms connected by single bonds, e.g., —(CH₂)ₓ—, wherein x is 2-5, and wherein one or more of the methylene groups may be replaced by O, S, S(O) or S(O)₂, and wherein the alkylene may further be substituted with one or more substituents selected from optionally substituted alkyl, cycloalkyl, aryl, including fused aryl where appropriate, heterocyclyl, oxo, halogen, hydroxy, carboxy, alkoxy, alkoxycarbonyl, and the like.

The term “cycloalkyl” refers to monocyclic, bicyclic or tricyclic hydrocarbon groups of 3-12 carbon atoms, each of which may contain one or more carbon-to-carbon double bonds.

The term “substituted cycloalkyl” refers to those cycloalkyl groups as described above substituted by one or more substituents, preferably 1-3, such as alkyl, halo, cyano, oxo, hydroxy, alkoxy, alkylamino, dialkylamino, alkylthio, sulfonyl, heterocyclyl, and the like.

Exemplary monocyclic hydrocarbon groups include, but are not limited to, cyclopropyl, cyclobutyl, cyclopentyl, cyclopentenyl, cyclohexyl, cyclohexenyl, 4,4-dimethylcyclohex-1-yl, cyclooctenyl, and the like.

Exemplary bicyclic hydrocarbon groups include bornyl, indyl, hexahydroindyl, tetrahydronaphthyl, decahydronaphthyi, bicyclo[2.1.1]hexyl, bicyclo[2.2.1]heptyl, bicyclo[2.2.1]heptenyl, 6,6-dimethylbicyclo[3.1.1]heptyl, 2,6,6-trimethylbicyclo[3.1.1]heptyl, bicyclo[2.2.2]octyl, and the like.

Exemplary tricyclic hydrocarbon groups include adamantyl and the like.

In the definitions listed herein, when a reference to an alkyl, cycloalkyl, alkenyl or alkynyl group is made as part of the term, a substituted alkyl, cycloalkyl, alkenyl or alkynyl group is also intended.

The term “alkoxy” refers to alkyl-O—.

The term “cycloalkoxy” refers to cycloalkyl-O—.

The term “alkanoyl” refers to alkyl-C(O)—.

The term “cycloalkanoyl” refers to cycloalkyl-C(O)—.

The term “alkenoyl” refers to alkenyl-C(O)—.

The term “alkynoyl” refers to alkynyl-C(O)—.

The term “alkanoyloxy” refers to alkyl-C(O)—O—.

The terms “alkylamino” and “dialkylamino” refer to alkyl-NH— and (alkyl)₂N—, respectively.

The term “alkanoylamino” refers to alkyl-C(O)—NH—.

The term “alkylthio” refers to alkyl-S—.

The term “trialkylsilyl” refers to (alkyl)₃Si—.

The term “trialkylsilyloxy” refers to (alkyl)₃SiO—.

The term “alkylthiono” refers to alkyl-S(O)—.

The term “alkylsulfonyl” refers to alkyl-S(O)₂—.

The term “alkoxycarbonyl” refers to alkyl-O—C(O)—.

The term “alkoxycarbonyloxy” refers to alkyl-O—C(O)O—.

The term “carbamoyl” refers to H₂NC(O)—, alkyl-NHC(O)—, (alkyl)₂NC(O)—, aryl-NHC(O)—, alkyl(aryl)-NC(O)—, heteroaryl-NHC(O)—, alkyl(heteroaryl)-NC(O)—, aralkyl-NHC(O)—, alkyl(aralkyl)-NC(O)— and the like.

The term “sulfamoyl” refers to H₂NS(O)₂—, alkyl-NHS(O)₂—, (alkyl)₂NS(O)₂—, aryl-NHS(O)₂—, alkyl(aryl)-NS(O)₂—, (aryl)₂NS(O)₂—, heteroaryl-NHS(O)₂—, aralkyl-NHS(O)₂—, heteroaralkyl-NHS(O)₂— and the like.

The term “sulfonamido” refers to alkyl-S(O)₂—NH—, aryl-S(O)₂—NH—, aralkyl-S(O)₂—NH—, heteroaryl-S(O)₂—NH—, heteroaralkyl-S(O)₂—NH—, alkyl-S(O)₂—N(alkyl)-, aryl-S(O)₂—N(alkyl)-, aralkyl-S(O)₂—N(alkyl)-, heteroaryl-S(O)₂—N(alkyl)-, heteroaralkyl-S(O)₂—N(alkyl)- and the like.

The term “sulfonyl” refers to alkylsulfonyl, arylsulfonyl, heteroarylsulfonyl, aralkylsulfonyl, heteroaralkylsulfonyl and the like.

The term “optionally substituted amino” refers to a primary or secondary amino group which may optionally be substituted by a substituent such as acyl, sulfonyl, alkoxycarbonyl, cycloalkoxycarbonyl, aryloxycarbonyl, heteroaryloxycarbonyl, aralkoxycarbonyl, heteroaralkoxycarbonyl, carbamoyl, and the like.

The term “aryl” refers to monocyclic or bicyclic aromatic hydrocarbon groups having 6-12 carbon atoms in the ring portion, such as phenyl, biphenyl, naphthyl, 2,3-dihydro-1H-indenyl and tetrahydronaphthyl.

The term “substituted aryl” refers to those aryl groups as described above substituted by 1-4 substituents in each ring portion, such as alkyl, trifluoromethyl, cycloalkyl, halo, hydroxy, alkoxy, methylenedioxy, acyl, alkanoyloxy, aryloxy, optionally substituted amino, thiol, alkylthio, arylthio, nitro, cyano, carboxy, alkoxycarbonyl, carbamoyl, aikyithiono, sulfonyl, sulfonamido, heterocyclyl, and the like.

The term “monocyclic aryl” refers to optionally substituted phenyl as described above under aryl. Preferably, the monocyclic aryl is substituted by 1-3 substituents selected from the group consisting of C₁₋₆ alkyl, C₁₋₆ alkoxy, halogen, cyano, or trifluoromethyl.

In the definitions listed herein, when a reference to an aryl group is made as part of the term, a substituted aryl group is also intended.

The term “aralkyl” refers to an aryl group bonded directly through an alkyl group, such as benzyl.

The term “aralkanoyl” refers to aralkyl-C(O)—.

The term “aralkylthio” refers to aralkyl-S—.

The term “aralkoxy” refers to an aryl group bonded directly through an alkoxy group.

The term “arylsulfonyl” refers to aryl-S(O)₂—.

The term “arylthio” refers to aryl-S—.

The term “aroyl” refers to aryl-C(O)—.

The term “aroyloxy” refers to aryl-C(O)—.

The term “aroylamino” refers to aryl-C(O)—NH—.

The term “aryloxycarbonyl” refers to aryl-O—C(O)—.

The term “heterocyclyl” or “heterocyclo” refers to fully saturated or unsaturated, aromatic or nonaromatic cyclic group, e.g., which is a 4- to 7-membered monocyclic, 7- to 12-membered bicyclic or 10- to 15-membered tricyclic ring system, which has at least one heteroatom in at least one carbon atom-containing ring. Each ring of the heterocyclic group containing a heteroatom may have 1, 2 or 3 heteroatoms selected from nitrogen atoms, oxygen atoms and sulfur atoms, where the nitrogen and sulfur heteroatoms may also optionally be oxidized. The heterocyclic group may be attached at a heteroatom or a carbon atom.

Exemplary monocyclic heterocyclic groups include pyrrolidinyl, pyrrolyl, pyrazolyl, oxetanyl, pyrazolinyl, imidazolyl, imidazolinyl, imidazolidinyl, triazolyl, oxazolyl, oxazolidinyl, isoxazolinyl, isoxazolyl, thiazolyl, thiadiazolyl, thiazolidinyl, isothiazolyl, isothiazolidinyl, furyl, tetrahydrofuryl, thienyl, oxadiazolyl, piperidinyl, piperazinyl, 2-oxopiperazinyl, 2-oxopiperidinyl, 2-oxopyrrolodinyl, 2-oxoazepinyl, azepinyl, 4-piperidonyl, pyridinyl (pyridyl), pyrazinyl, pyrimidinyl, pyridazinyl, tetrahydropyranyl, morpholinyl, thiamorpholinyl, thiamorpholinyl sulfoxide, thiamorpholinyl sulfone, 1,3-dioxolane and tetrahydro-1,1-dioxothienyl, 1,1,4-trioxo-1,2,5-thiadiazolidin-2-yl, and the like.

Exemplary bicyclic heterocyclic groups include indolyl, dihydroidolyl, benzothiazolyl, benzoxazinyl, benzoxazolyl, benzothienyl, benzothiazinyl, quinuclidinyl, quinolinyl, tetrahydroquinolinyl, decahydroquinolinyl, isoquinolinyl, tetrahydroisoquinolinyl, decahydroisoquinolinyl, benzimidazolyl, benzopyranyl, indolizinyl, benzofuryl, chromonyl, coumarinyl, benzopyranyl, cinnolinyl, quinoxalinyl, indazolyl, pyrrolopyridyl, furopyridinyl (such as furo[2,3-c]pyridinyl, furo[3,2-b]pyridinyl or furo[2,3-b]pyridinyl), dihydroisoindolyl, 1,3-dioxo-1,3-dihydroisoindol-2-yl, dihydroquinazolinyl (such as 3,4-dihydro-4-oxo-quinazolinyl), phthalazinyl, and the like.

Exemplary tricyclic heterocyclic groups include carbazolyl, dibenzoazepinyl, dithienoazepinyl, benzindolyl, phenanthrolinyl, acridinyl, phenanthridinyl, phenoxazinyl, phenothiazinyl, xanthenyl, carbolinyl, and the like.

The term “substituted heterocyclyl” refers to those heterocyclic groups described above substituted with 1, 2 or 3 substituents selected from the group consisting of the following:

(a) alkyl;

(b) hydroxyl (or protected hydroxyl);

(c) halo;

(d) oxo, i.e., ═O;

(e) optionally substituted amino;

(f) alkoxy;

(g) cycloalkyl;

(h) carboxy;

(i) heterocyclooxy;

(j) alkoxycarbonyl, such as unsubstituted lower alkoxycarbonyl;

(k) thiol;

(l) nitro;

(m) cyano;

(n) sulfamoyl;

(o) alkanoyloxy;

(p) aroyloxy;

(q) arylthio;

(r) aryloxy;

(s) alkylthio;

(t) formyl;

(u) carbamoyl;

(v) aralkyl; and

(w) aryl optionally substituted with alkyl, cycloalkyl, alkoxy, hydroxyl, amino, acylamino, alkylamino, dialkylamino or halo.

The term “heterocyclooxy” denotes a heterocyclic group bonded through an oxygen bridge.

The term “heterocycloalkyl” refers to nonaromatic heterocyclic groups as described above.

The term “heteroaryl” refers to an aromatic heterocycle, e.g., monocyclic or bicyclic aryl, such as pyrrolyl, pyrazolyl, imidazolyl, triazolyl, oxazolyl, isoxazolyl, thiazolyl, isothiazolyl, furyl, thienyl, pyridyl, pyrazinyl, pyrimidinyl, pyridazinyl, indolyl, benzothiazolyl, benzoxazolyl, benzothienyl, quinolinyl, isoquinolinyl, benzimidazolyl, benzofuryl and the like, optionally substituted by, e.g., halogen, cyano, nitro, trifluoromethyl, lower alkyl, or lower alkoxy.

The term “heterocycloalkanoyl” refers to heterocycloalkyl-C(O)—.

The term “heteroarylsulfonyl” refers to heteroaryl-S(O)₂—.

The term “heteroaroyl” refers to heteroaryl-C(O)—.

The term “heteroaroylamino” refers to heteroaryl-C(O)NH—.

The term “heteroaralkyl” refers to a heteroaryl group bonded through an alkyl group.

The term “heteroaralkanoyl” refers to heteroaralkyl-C(O)—.

The term “heteroaralkanoylamino” refers to heteroaralkyl-C(O)NH—.

The term “acyl” refers to alkanoyl, cycloalkanoyl, alkenoyl, alkynoyl, aroyl, heterocycloalkanoyl, heteroaroyl, aralkanoyl, heteroaralkanoyl, and the like.

The term “substituted acyl” refers to those acyl groups described above wherein the alkyl, cycloalkyl, alkenyl, alkynyl, aryl, heterocycloalkyl, heteroaryl, aralkyl, or heteroaralkyl group is substituted as described herein above respectively.

The term “acylamino” refers to alkanoylamino, aroylamino, heteroaroylamino, aralkanoylamino, heteroaralkanoylamino, and the like.

The term “halogen” or “halo” refers to fluorine, chlorine, bromine and iodine.

Pharmaceutically acceptable salts of the compounds of the present invention refer to salts formed with acids, namely acid addition salts, such as of mineral acids, organic carboxylic acids and organic sulfonic acids, e.g., hydrochloric acid, maleic acid and methanesulfonic acid, respectively.

Similarly, pharmaceutically acceptable salts of the compounds of the invention refer to salts formed with bases, namely cationic salts, such as alkali and alkaline earth metal salts, e.g., sodium, lithium, potassium, calcium and magnesium, as well as ammonium salts, e.g., ammonium, trimethylammonium, diethylammonium and tris(hydroxymethyl)-methyl-ammonium salts and salts with amino acids provided an acidic group constitutes part of the structure.

As described herein above, the present invention provides 2-aminothiophene derivatives of formula (i), pharmaceutical compositions containing them, methods for preparing said compounds, and methods of treating conditions mediated by the A₁ adenosine receptor including, but not limited to, pain, in particular, chronic pain such as neuropathic pain, and inflammatory pain, cardiac disease or disorder such as congestive heart failure, cardiac disarrhythmias, e.g., peroxysmal supraventricular, tachycardia, angina, myocardial infarction and stroke, neurological disease or injury, sleep disorders, epilepsy, depression, and various inflammatory conditions, by administration of a therapeutically effective amount of a compound of the present invention, or a pharmaceutical composition thereof.

Preferred are the compounds of formula (I) having the formula

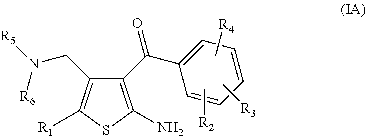

wherein
R₁ is hydrogen, alkyl, substituted alkyl, cycloalkyl, substituted cycloalkyl, alkenyl, substituted alkenyl, alkynyl, substituted alkynyl, aryl, substituted aryl, heteroaryl, or substituted heteroaryl;
R₂, R₃, and R₄ are, independently from each other, hydrogen, alkyl, substituted alkyl, aryl, substituted aryl, cycloalkyl, substituted cycloalkyl, halogen, hydroxyl, nitro, cyano, alkoxy, or substituted alkoxy; or
R₂ and R₃ combined are alkylene which together with the carbon atoms to which they are attached form a 4- to 7-membered fused ring, provided that R₂ and R₃ are attached to carbon atoms adjacent to each other;
R₅ and R₆ are, independently from each other, hydrogen, alkyl, substituted alkyl, aryl, substituted aryl, cycloalkyl, substituted cycloalkyl, heterocyclyl, or substituted heterocyclyl;
   or a pharmaceutically acceptable salt thereof.

Further preferred are the compounds of formula (I) having the formula

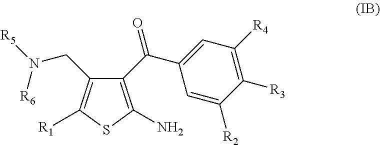

wherein
R₁ is hydrogen, alkyl, substituted alkyl, cycloalkyl, substituted cycloalkyl, alkenyl, substituted alkenyl, alkynyl, substituted alkynyl, aryl, substituted aryl, heteroaryl, or substituted heteroaryl;
R₂, R₃, and R₄ are, independently from each other, hydrogen, alkyl, substituted alkyl, aryl, substituted aryl, cycloalkyl, substituted cycloalkyl, halogen, hydroxyl, nitro, cyano, alkoxy, or substituted alkoxy;
R₅ and R₆ are, independently from each other, hydrogen, alkyl, substituted alkyl, aryl, substituted aryl, cycloalkyl, substituted cycloalkyl, heterocyclyl, or substituted heterocyclyl.

Preferred are the compounds of formula (IB), designated as the A group, wherein
R₁ is hydrogen, alkyl, substituted alkyl, aryl, or substituted aryl;
   or a pharmaceutically acceptable salt thereof.

Preferred are the compounds in the A group wherein
R₁ is monocyclic aryl, or substituted monocyclic aryl;
   or a pharmaceutically acceptable salt thereof.

Preferred are also the compounds in the A group wherein
R₅ and R₆ are, independently from each other, alkyl, substituted alkyl, cycloalkyl, or substituted cycloalkyl;
   or a pharmaceutically acceptable salt thereof.

Preferred are also the compounds in the A group, designated as the B group, wherein
R₂ and R₄ are hydrogen;
   or a pharmaceutically acceptable salt thereof.

Preferred are the compounds in the B group wherein
R₃ is halogen, cyano, or trifluoromethyl;
   or a pharmaceutically acceptable salt thereof.

Preferred are also the compounds in the A group, designated as the C group, wherein
R₂ and R₃ are hydrogen;
   or a pharmaceutically acceptable salt thereof.

Preferred are the compounds in the C group wherein
R₄ is halogen, cyano, or trifluoromethyl;
   or a pharmaceutically acceptable salt thereof.

The compounds of the invention depending on the nature of the substituents may possess one or more asymmetric centers. The resulting diastereoisomers, optical isomers, i.e., enantiomers, and geometric isomers, and mixtures thereof, are encompassed by the instant invention.

Particular embodiments of the invention are:
{2-Amino-4-[(diisopropylamino)methyl]-5-phenylthiophen-3-yl}(4-chlorophenyl)methanone;
{2-Amino-4-[(dicyclohexylamino)methyl]-5-phenylthiophen-3-yl}(4-chlorophenyl)methanone;
{2-Amino-4-[(diethylamino)methyl]-5-phenylthiophen-3-yl}(4-chlorophenyl)methanone;
{2-Amino-4-[(diallylamino)methyl]-5-phenylthiophen-3-yl}(4-chlorophenyl)methanone;
{2-Amino-4-[(dipropylamino)methyl]-5-phenylthiophen-3-yl}(4-chlorophenyl)methanone;
{2-Amino-4-[(t-butyl(methyl)amino)methyl]-5-phenylthiophen-3-yl}(4-chlorophenyl)methanone;
{2-Amino-4-[benzyl(methyl)amino)methyl]-5-phenylthiophen-3-yl}(4-chlorophenyl)methanone;
{2-Amino-4-[(dimethylamino)methyl]-5-phenylthiophen-3-yl}(4-chlorophenyl)methanone;
{2-Amino-4-[(butyl(methyl)amino)methyl]-5-phenylthiophen-3-yl}(4-chlorophenyl)methanone;
{2-Amino-5-(4-cyanophenyl)-4-[(dicyclohexylamino)methyl]thiophen-3-yl}(4-chlorophenyl)methanone;
{2-Amino-5-(4-t-butylphenyl)-4-[(dicyclohexylamino)methyl]thiophen-3-yl}(4-chlorophenyl)methanone;
{2-Amino-5-(4-chlorophenyl)-4-[(dicyclohexylamino)methyl]thiophen-3-yl}(4-chlorophenyl)methanone;
{2-Amino-4-[(dicyclohexylamino)methyl]-5-(2-fluorophenyl)thiophen-3-yl}(4-chlorophenyl)methanone;
{2-Amino-4-[(dicyclohexylamino)methyl]-5-(3-fluorophenyl)thiophen-3-yl}(4-chlorophenyl)methanone;
{2-Amino-4-[(dicyclohexylamino)methyl]-5-(4-fluorophenyl)thiophen-3-yl}(4-chlorophenyl)methanone;
{2-Amino-4-[(dibutylamino)methyl]-5-phenylthiophen-3-yl}(4-chlorophenyl)methanone;
{2-Amino-4-[(diisobutylamino)methyl]-5-phenylthiophen-3-yl}(4-chlorophenyl)methanone;
{2-Amino-4-[(dipentylamino)methyl]-5-phenylthiophen-3-yl}(4-chlorophenyl)methanone;
{2-Amino-4-[(dicyclopentylamino)methyl]-5-phenylthiophen-3-yl}(4-chlorophenyl)methanone;
{2-Amino-4-[(cyclohexyl(propyl)amino)methyl]-5-phenylthiophen-3-yl}(4-chlorophenyl)methanone;
{4-[(Allyl(cyclohexyl)amino)methyl]-2-amino-5-phenylthiophen-3-yl)}(4-chlorophenyl)methanone;
{4-[(2-methoxy-2-oxoethyl)methylamino)methyl]-2-amino-5-phenylthiophen-3-yl)}(4-chlorophenyl)methanone;
{2-Amino-4-[(bis(2-methoxyethyl)amino)methyl]-5-phenylthiophen-3-yl}(4-chlorophenyl)methanone;
{2-Amino-4-[(dineopentylamino)methyl]-5-phenylthiophen-3-yl}(4-chlorophenyl)methanone;
{2-Amino-4-[(1-adamantylamino)methyl]-5-phenylthiophen-3-yl}(4-chlorophenyl)methanone;
{2-Amino-4-[((3,4-dichlorophenyl)(methyl)amino)methyl]-5-phenylthiophen-3-yl}(4-chlorophenyl)methanone;
{2-Amino-4-[((4-methoxyphenyl)(methyl)amino)methyl]-5-phenylthiophen-3-yl}(4-chlorophenyl)methanone;
{2-Amino-4-[((4-methylphenyl)(methyl)amino)methyl]-5-phenylthiophen-3-yl}(4-chlorophenyl)methanone;
{2-Amino-5-(3,4-dichlorophenyl)-4-[(dicyclohexylamino)methyl]thiophen-3-yl}(4-chlorophenyl)methanone;
{2-Amino-4-[(dicyclohexylamino)methyl]-5-(4-isopropoxyphenyl)thiophen-3-yl}(4-chlorophenyl)methanone;
{2-Amino-4-[(dicyclohexylamino)methyl]-5-(3,5-difluorophenyl)thiophen-3-yl}(4-chlorophenyl)methanone;
{2-Amino-4-[(dicyclohexylamino)methyl]-5-(4-methylphenyl)thiophen-3-yl}(4-chlorophenyl)methanone;
{2-Amino-4-[(dicyclohexylamino)methyl]-5-(4-methoxyphenyl)thiophen-3-yl}(4-chlorophenyl)methanone;
{2-Amino-4-[(diethylamino)methyl]thiophen-3-yl}(4-chlorophenyl)methanone;
{2-Amino-4-[(dipropylamino)methyl]thiophen-3-yl}(4-chlorophenyl)methanone;
{2-Amino-4-[(diisopropylamino)methyl]thiophen-3-yl}(4-chlorophenyl)methanone;
{2-Amino-4-[(methyl(phenyl)amino)methyl]thiophen-3-yl}(4-chlorophenyl)methanone;
{2-Amino-4-[(ethyl(phenyl)amino)methyl]thiophen-3-yl}(4-chlorophenyl)methanone;
{2-Amino-4-[(4-fluorophenyl(methyl)amino)methyl]thiophen-3-yl}(4-chlorophenyl)methanone;
{2-Amino-4-[(4-chlorophenyl(methyl)amino)methyl]thiophen-3-yl}(4-chlorophenyl)methanone;
{2-Amino-4-[(methyl(4-trifluoromethylphenyl)amino)methyl]thiophen-3-yl}(4-chlorophenyl)methanone;
{2-Amino-4-[(benzyl(methyl)amino)methyl]-5-methylthiophen-3-yl}(4-chlorophenyl)methanone;
{2-Amino-[(dicyclohexylamino)methyl]-5-(furan-3-yl)thiophen-3-yl}(4-chlorophenyl)methanone;
{2-Amino-4-[(dicyclohexylamino)methyl]-5-(thiophen-2-yl)thiophen-3-yl}(4-chlorophenyl)methanone;
{2-Amino-4-[(dicyclohexylamino)methyl]-5-(furan-2-yl)thiophen-3-yl}(4-chlorophenyl)methanone;
{5-Amino-5′-chloro-3-[(dicyclohexylamino)methyl]-2,2′-bithiophen-4-yl}(4-chlorophenyl)methanone;
(E)-{2-amino-5-(3-cyclohexylprop-1-enyl)-4-[(dicyclohexylamino)methyl]thiophen-3-yl}(4-chlorophenyl)methanone;
(E)-{2-Amino-5-[3-(4-chlorophenyl)prop-1-enyl]-4-[(dicyclohexylamino)methyl]thiophen-3-yl}(4-chlorophenyl)methanone;
{2-Amino-4-[(dicyclohexylamino)methyl]-5-[4-(2-methoxyethoxy)phenyl]thiophen-3-yl}(4-chlorophenyl)methanone;
{2-amino-4-[(dicyclohexylamino)methyl]-5-(pyridin-2-yl)thiophen-3-yl}(4-chlorophenyl)methanone;
{2-amino-4-[(dicyclohexylamino)methyl]-5-(pyridin-3-yl)thiophen-3-yl}(4-chlorophenyl)methanone; and
{2-amino-4-[(dicyclohexylamino)methyl]-5-(pyridin-4-yl)thiophen-3-yl}(4-chlorophenyl)methanone;
   or a pharmaceutically acceptable salt thereof.

Compounds of formula (I) may be prepared using methods well known in the art, or using modifications thereof, e.g., as outlined below in Schemes 1 to 6.

As exemplified in Scheme 1, compounds of formula (I) wherein R₁ is hydrogen, and W, R₅ and R₆ have a meaning as defined herein above, i.e., compounds of formula (I′), may be prepared by condensing a compound of formula (II) wherein W has a meaning as defined herein above, with 2,5-dimethyl-[1,4]dithiane-2,5-diol of formula (II′) in the presence of a base such as triethylamine (TEA), diisopropylethylamine (DIEA), N-methylmorpholine (NMM) or morpholine, in an organic solvent such as a lower alcohol, preferably, ethanol (EtOH) or isopropanol, to afford a compound of formula (III) wherein W has a meaning as defined herein above.

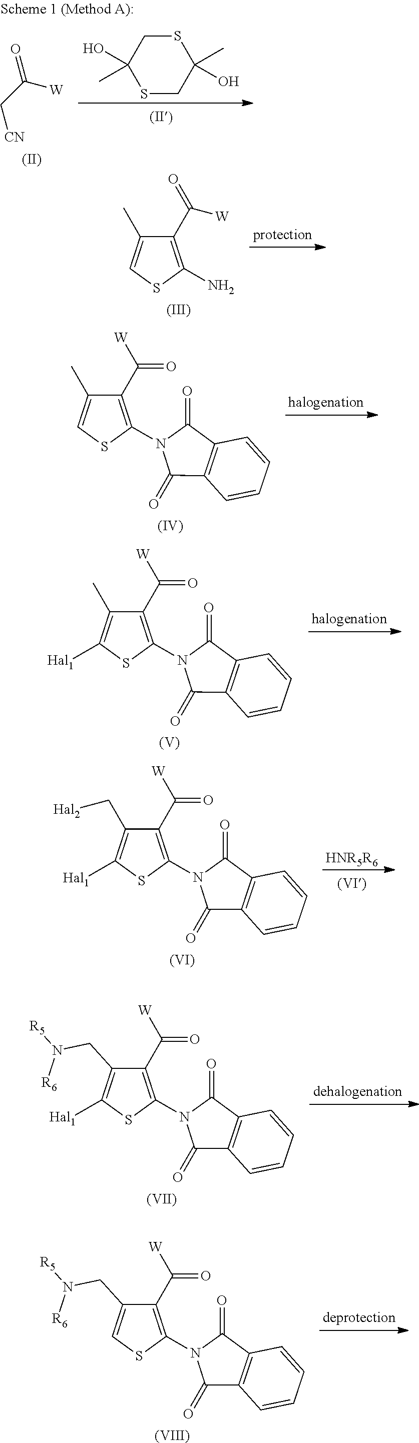

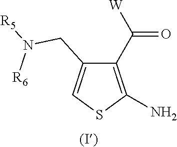

Compounds of formula (II) are known, or if they are novel they may be prepared using methods well known in the art, or modifications thereof, e.g., as described in U.S. Pat. No. 6,323,214.

A resulting compound of formula (III) may then be converted to a compound of formula (IV) wherein W has a meaning as defined herein above, and the amino group has been protected as a phthalimido group, under reaction conditions well known in the art, e.g., by treating a compound of formula (III) with phthalic anhydride in the presence of an acid, such as acetic acid, at an elevated temperature.

A resulting compound of formula (IV) may then be halogenated at the 5-position of the thiophene ring to afford a compound of formula (V) wherein W has a meaning as defined herein above, and Hal₁ represents chloride, bromide or iodide, using methods well known in the art, e.g., a compound of formula of formula (IV) may be treated with a halogenating agent such as N-halosuccinimide, e.g., N-bromosuccinimide (NBS), in the presence of a catalyst such as benzoyl peroxide, and an inert organic solvent, such as an aromatic hydrocarbon, e.g., benzene, to afford a compound of formula (V) wherein Hal₁ is, e.g., bromide.

Subsequent reaction of a resulting compound of formula (V) with a halogenating agent such as N-halosuccinimide, e.g., NBS, in the presence of a catalyst such as benzoyl peroxide and an organic solvent such as a halogenated hydrocarbon, e.g., carbontetrachloride or 1,2-dichloroethane, affords a compound of formula (VI) wherein W has a meaning as defined herein above, and Hal₁ and Hal₂ represent, independently from each other, chloride, bromide or iodide.

A resulting compound of formula (VI) may then be coupled with an amine of formula (VI′) wherein R₅ and R₆ have a meaning as defined herein above, in the presence of a base such as TEA, DIEA, NMM, or potassium or cesium carbonate, and an appropriate organic solvent, such as dichloromethane (DCM), chloroform (CHCl₃) and N,N-dimethylformamide (DMF), to afford a compound of formula (VII) wherein W, R₅, R₆ and Hal₁ have a meaning as defined herein above.

Amines of formula (VI′) are known, or if they are novel they may be prepared using methods well known in the art, or modifications thereof.

A resulting compound of formula (VII) may then be dehalogenated in the presence of a reducing agent, e.g., molecular hydrogen in the presence of a catalyst such as palladium on carbon, and an organic solvent, such as ethyl acetate (EtOAc), a lower alcohol, e.g., EtOH and methanol (MeOH), tetrahydrofuran (THF) or DMF, to afford a compound of formula (VIII) wherein W, R₅ and R₆ have a meaning as defined herein above. Preferably, the dehalogenation is conducted in the presence of an extrinsic base, e.g., TEA.

Finally, a compound of formula (VIII) may be converted to a compound of formula (I′) wherein W, R₅ and R₆ have a meaning as defined herein above, by removal of the phthalimido protecting group, e.g., by treatment with hydrazine or ethylenediamine in an organic solvent such as lower alcohol, e.g., EtOH.

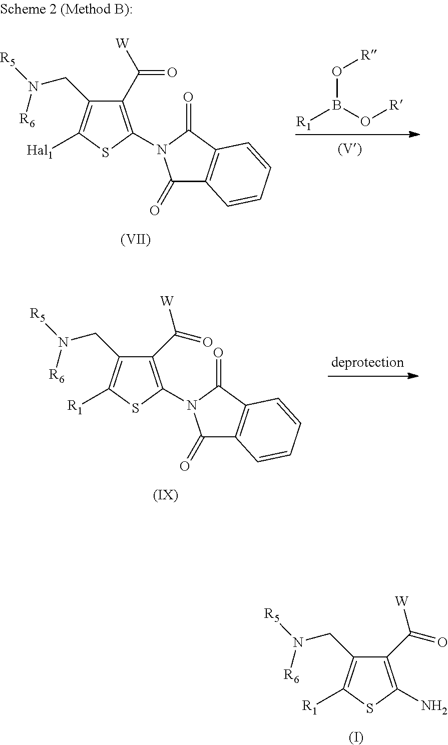

As outlined in Scheme 2, compounds of formula (VII) wherein W, R₅ and R₆ have a meaning as defined herein above, may be coupled with a compound of the formula (V′) wherein R₁ is alkenyl, substituted alkenyl, alkynyl, substituted alkynyl, aryl, substituted aryl, heteroaryl or substituted heteroaryl, and R′ and R″ are hydrogen or lower alkyl, or R′ and R″ combined are alkylene which together with the boron and the oxygen atoms form a 5- or 6-membered ring, in the presence of a catalyst, preferably a palladium catalyst, e.g., palladium(II)acetate, [1,1-bis(diphenylphosphino)ferrocene]dichloropalladium(II) methylene chloride complex, or tetrakis(triphenylphosphine)palladium(0), and a base such as sodium hydroxide (NaOH), cesium fluoride, or sodium, potassium or cesium carbonate, in an appropriate solvent, e.g., acetonitrile (ACN), DMF, dimethoxyethane (DME), 1,4-dioxane or toluene, or a mixture of solvents thereof, to afford a compound of formula (IX) wherein R₁ is alkenyl, substituted alkenyl, alkynyl, substituted alkynyl, aryl, substituted aryl, heteroaryl or substituted heteroaryl. Preferably, R′ and R″ are hydrogen, and the above coupling reaction, i.e., Suzuki reaction, is conducted in toluene or 1,4-dioxane in the presence of [1,1-bis(diphenylphosphino)ferrocene]dichloropalladium(II) methylene chloride complex, and cesium fluoride at a temperature close to the boiling point of the solvent.

Compounds of formula (V′) are known, or if they are novel they may be prepared using methods well known in the art, e.g., as described herein in the illustrative Examples, or modifications thereof.

As exemplified in Scheme 3, compounds of formula (I) wherein R₁ is alkyl, substituted alkyl, cycloalkyl, substituted cycloalkyl, aryl, substituted aryl, heteroaryl, or substituted heteroaryl, and W, R₅, and R₆ have a meaning as defined herein above may be prepared by the reaction of a compound of formula (II) wherein W has a meaning as defined herein above, with a ketone of formula (X) wherein R₁ is alkyl, substituted alkyl, cycloalkyl, substituted cycloalkyl, aryl, substituted aryl, heteroaryl, or substituted heteroaryl, in the presence of elemental sulfur and an appropriate base, such as TEA, DIEA, NMM, or morpholine, preferably, morpholine, in an organic solvent such as a lower alcohol, preferably EtOH, to afford a compound of formula (III′), wherein R₁ is alkyl, substituted alkyl, cycloalkyl, substituted cycloalkyl, aryl, substituted aryl, heteroaryl, or substituted heteroaryl, and W has a meaning as defined herein above.

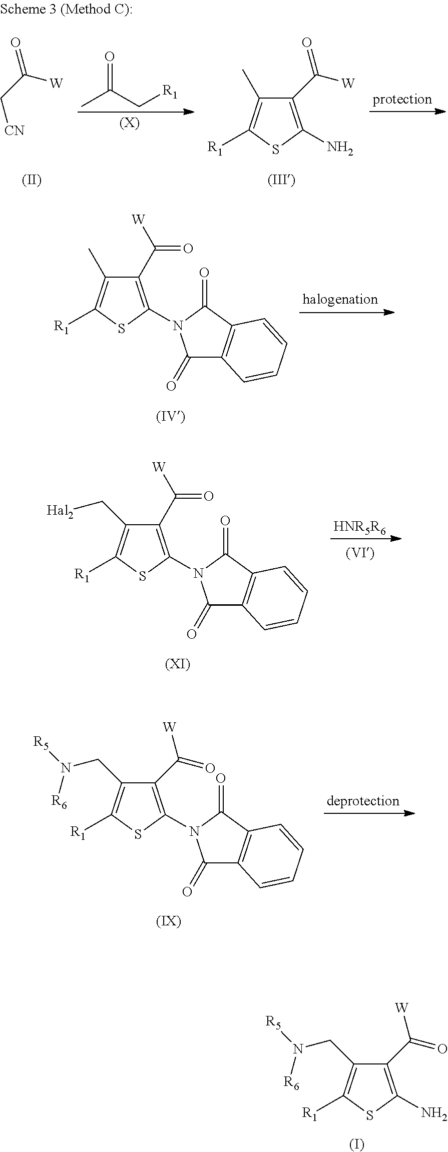

Compounds of formula (X) are known, or if they are novel they may be prepared using methods well known in the art, or modifications thereof.

A resulting compound of formula (III′) may then be converted to a compound (IV′) wherein R₁ is alkyl, substituted alkyl, substituted alkyl, cycloalkyl, substituted cycloalkyl, aryl, substituted aryl, heteroaryl, or substituted heteroaryl, and W has a meaning as defined herein above, by treating a compound (III′) with phthalic anhydride in the presence of an acid, such as acetic acid, at an elevated temperature.

A resulting compound of formula (IV′) may then be halogenated on the methyl group at the 4-position of the thiophene ring to afford a compound of formula (XI) wherein R₁ is alkyl, substituted alkyl, cycloalkyl, substituted cycloalkyl, aryl, substituted aryl, heteroaryl, or substituted heteroaryl, W has a meaning as defined herein above, and Hal₂ represents chloride, bromide, or iodide, using methods well known in the art, e.g., by the reaction of a compound of formula (IV′) with a halogenating agent, such as an N-halosuccinimide, e.g. NBS, in the presence of a catalyst such as benzoyl peroxide, and an organic solvent, such as ACN, or a halogenated hydrocarbon, e.g., carbon tetrachloride, or 1,2-dichloroethane. It should be noted that the halogenation of the methyl group at the 4-position of the thiophene ring of compounds of formula (IV′) may be conducted in the absence of a catalyst when ACN is employed as the solvent.

A resulting compound of formula (XI) may then be coupled with an amine of formula (VI′) wherein R₅ and R₆ have a meaning as defined herein above, in the presence of a base such as TEA, DIEA, NMM, or potassium or cesium carbonate, and an appropriate organic solvent such as DCM, CHCl₃ and DMF, to afford a compound of formula (IX) wherein R₁ is alkyl, substituted alkyl, cycloalkyl, substituted cycloalkyl, aryl, substituted aryl, heteroaryl, or substituted heteroaryl, and W, R₅ and R₆ have a meaning as defined herein above.

Finally, a compound of formula (IX) may be converted to a compound of formula (I) wherein R₁ is alkyl, substituted alkyl, cycloalkyl, substituted cycloalkyl, aryl, substituted aryl, heteroaryl, or substituted heteroaryl, and W, R₅ and R₆ have a meaning as defined herein above, by removal of the phthalimido protecting group as described herein above.

Alternatively, as illustrated in Scheme 4, compounds of formula (IV′) wherein R₁ is alkenyl, substituted alkenyl, alkynyl, substituted alkynyl, aryl, substituted aryl, heteroaryl or substituted heteroaryl, and W has a meaning as defined herein above, may be obtained by coupling a compound of formula (V) wherein Hal₁ and W have a meaning as defined herein above, in the presence of a catalyst, preferably, a palladium catalyst, e.g., palladium(II)acetate, [1,1-bis(diphenylphosphino)ferrocene]dichloropalladium(II) methylene chloride complex, or tetrakis(triphenylphosphine)palladium(0), and a base such as sodium hydroxide (NaOH), cesium fluoride, or sodium, potassium or cesium carbonate, in an appropriate solvent, e.g., ACN, DMF, dimethoxyethane (DME), 1,4-dioxane or toluene, or a mixture of solvents thereof, with a compound of the formula (V′) wherein R₁ is alkenyl, substituted alkenyl, alkynyl, substituted alkynyl, aryl, substituted aryl, heteroaryl or substituted heteroaryl, and R′ and R″ are hydrogen or lower alkyl, or R′ and R″ combined are alkylene which together with the boron and the oxygen atoms form a 5- or 6-membered ring, to afford a compound of formula (IV′) wherein R₁ is alkenyl, substituted alkenyl, alkynyl, substituted alkynyl, aryl, substituted aryl, heteroaryl or substituted heteroaryl. Preferably, R′ and R″ are hydrogen, and the above coupling reaction, i.e., Suzuki reaction, is conducted in toluene or 1,4-dioxane in the presence of [1,1′-bis(diphenylphosphino)-ferrocene]dichloropalladium(II) methylene chloride complex and cesium fluoride at a temperature close to the boiling point of the solvent.

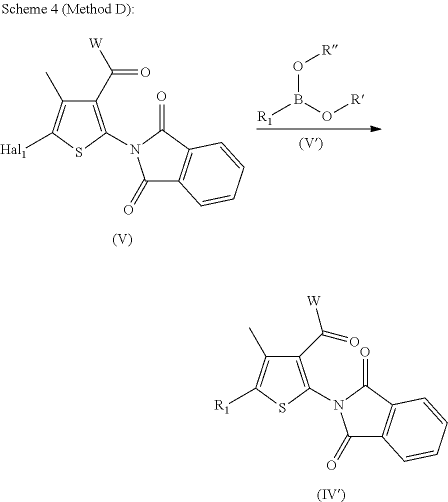

The remaining steps are then carried out as described herein above in Scheme 3 to afford compounds of formula (I) wherein R₁ is alkenyl, substituted alkenyl, alkynyl, substituted alkynyl, aryl, substituted aryl, heteroaryl or substituted heteroaryl, and W, R₅ and R₆ have a meaning as defined herein above.

Likewise, as illustrated in Scheme 5, compounds of formula (V) wherein Hal₁ and W have a meaning as defined herein above, may be converted to compounds of formula (IV′) wherein R₁ is alkenyl, substituted alkenyl, alkynyl, substituted alkynyl, aryl, substituted aryl, heteroaryl or substituted heteroaryl, and W has a meaning as defined herein above, by reacting a compound of formula (V) with a compound of formula (V″) wherein R₁ is alkenyl, substituted alkenyl, alkynyl, substituted alkynyl, aryl, substituted aryl, heteroaryl or substituted heteroaryl, and R represents lower alkyl, in the presence of a catalyst, preferably a palladium catalyst, e.g., palladium(II)acetate, dichloropalladium(II) bis(triphenylphosphine), Pd₂(dba)₃ (dibenzylideneacetone) or tetrakis(triphenylphosphine)palladium(0), and optionally in the presence of an additive such as cesium fluoride, potassium fluoride tetrabutylammonium fluoride, copper(I) iodide or lithium chloride, in an appropriate solvent, e.g. 1,4-dioxane, DMF, THF, ACN or hexamethylphosphoroustriamide (HMPT), or a mixture of solvents thereof. Preferably, R in compounds of formula (V″) is n-butyl, and the above described coupling reaction, i.e., Stille coupling, is conducted in 1,4-dioxane in the presence of lithium chloride and dichloropalladium(II) bis(triphenylphosphine) at a temperature close to the boiling point of the solvent.

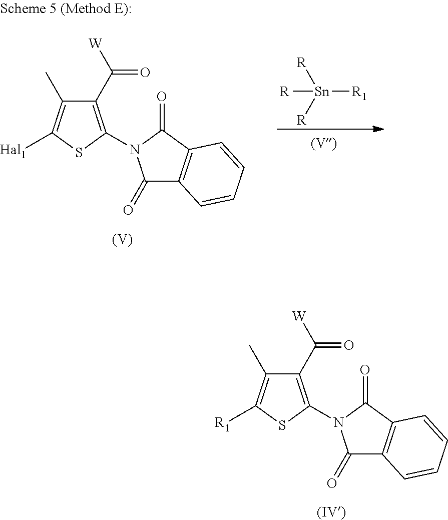

Compounds of formula (V″) are known, or if they are novel they may be prepared using methods well known in the art, e.g., as described herein in the illustrative Examples, or using modifications thereof.

In yet another approach, compounds of formula (IX) wherein R₁ is alkenyl, substituted alkenyl, alkynyl, substituted alkynyl, aryl, substituted aryl, heteroaryl or substituted heteroaryl, and R₅, R₆, and W have meanings as defined herein above, may be obtained as illustrated in Scheme 6. A compound of formula (VI) wherein Hal₁, Hal₂, and W have a meaning as defined herein above may be hydrolyzed by the treatment of a suitable aqueous base, such as aqueous sodium bicarbonate, sodium carbonate, NaOH, or potassium hydroxide, in the presence of a water miscible organic solvent, preferably THF, to afford a compound of formula (XII), wherein Hal₁ and W have meanings as defined above.

A resulting compound of formula (XII) may then be converted to a compound of formula (XIII) wherein R₁ is alkenyl, substituted alkenyl, alkynyl, substituted alkynyl, aryl, substituted aryl, heteroaryl or substituted heteroaryl, and W have a meaning as defined herein above, by coupling a compound of formula (XII) with a compound of formula (V′) wherein R₁ is alkenyl, substituted alkenyl, alkynyl, substituted alkynyl, aryl, substituted aryl, heteroaryl or substituted heteroaryl, and R′ and R″ are hydrogen or lower alkyl, or R′ and R″ combined are alkylene which together with the boron and the oxygen atoms form a 5- or 6-membered ring. The coupling of compounds of formula (XII) and (V′) may be effected by the presence of a catalyst, preferably a palladium catalyst, e.g., palladium(II)acetate, [1,1′-bis(diphenylphosphino)ferrocene]-dichloropalladium(II) methylene chloride complex, or tetrakis(triphenylphosphine)palladium(0), and a base such as NaOH, cesium fluoride, or sodium, potassium, or cesium carbonate, in an appropriate solvent, such as ACN, DMF, DME, 1,4-dioxane, DCM, or toluene, or a mixture of solvents thereof.

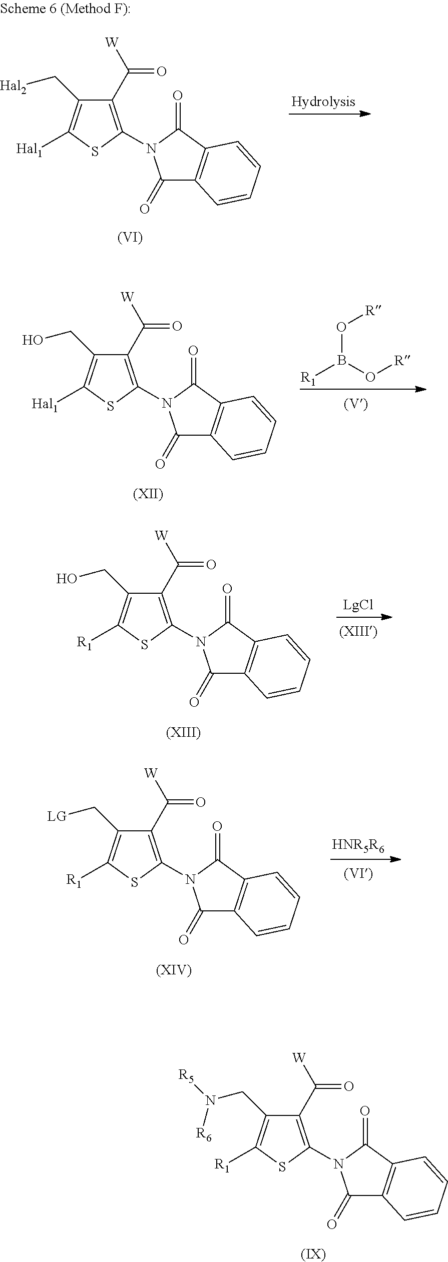

A resulting compound of formula (XIII) may then be converted to a compound of formula (XIV) wherein is R₁ is alkenyl, substituted alkenyl, alkynyl, substituted alkynyl, aryl, substituted aryl, heteroaryl or substituted heteroaryl, Lg represents a leaving group, such as p-toluenesulfonate, methanesulfonate or trifluoromethanesulfonate, preferably methanesulfonate, and W have a meaning as defined herein above, by the treatment with a compound of formula (XIII′) wherein Lg has a meaning as defined herein above, in the presence of an appropriate solvent, such as DME, DCM, 1,4-dioxane, THF, or CHCl₃, and a base such as TEA, trimethylamine, NMM, diethylisopropylamine, DIEA, triisopropylamine, or N-methylpiperidine.

A subsequent reaction of a resulting compound of formula (XIV) wherein R₁ is alkenyl, substituted alkenyl, alkynyl, substituted alkynyl, aryl, substituted aryl, heteroaryl or substituted heteroaryl, and Lg and W have a meaning as defined herein above, with an amine of formula (VI′) wherein R₅ and R₆ have a meaning as defined above, in the presence of a base such as TEA, DIEA, NMM, or potassium or cesium carbonate, and an appropriate solvent, such as DCM, CHCl₃, or DMF, affords a compound of formula (IX) wherein R₁, R₅, R₆ and W have a meaning as defined herein above.

The remaining deprotection step may then be performed as described herein above in Schemes 1 to 3, and affords a compound of formula (I) wherein R₁ is alkenyl, substituted alkenyl, alkynyl, substituted alkynyl, aryl, substituted aryl, heteroaryl or substituted heteroaryl, and R₅, R₆ and W have a meaning as defined herein above.

The processes described herein above may be conducted under inert atmosphere, preferably under nitrogen or argon atmosphere.

In starting compounds and intermediates which are converted to the compounds of the present invention in a manner described herein, functional groups present, such as amino, thiol, carboxyl and hydroxyl groups, are optionally protected by conventional protecting groups that are common in preparative organic chemistry. Protected amino, thiol, carboxyl and hydroxyl groups are those that can be converted under mild conditions into free amino thiol, carboxyl and hydroxyl groups without the molecular framework being destroyed or other undesired side reactions taking place.

The purpose of introducing protecting groups is to protect the functional groups from undesired reactions with reaction components under the conditions used for carrying out a desired chemical transformation. The need and choice of protecting groups for a particular reaction is known to those skilled in the art and depends on the nature of the functional group to be protected (hydroxyl group, amino group, etc.), the structure and stability of the molecule of which the substituent is a part and the reaction conditions.

Well-known protecting groups that meet these conditions and their introduction and removal are described, e.g., in McOmie, “Protective Groups in Organic Chemistry”, Plenum Press, London, NY (1973); and Greene and Wuts, “Protective Groups in Organic Synthesis”, 4th edition, John Wiley and Sons, Inc., NY (2007).

The above-mentioned reactions are carried out according to standard methods, in the presence or absence of diluent, preferably, such as are inert to the reagents and are solvents thereof, of catalysts, condensing or said other agents, respectively and/or inert atmospheres, at low temperatures, room temperature (RT) or elevated temperatures, preferably at or near the boiling point of the solvents used, and at atmospheric or super-atmospheric pressure. The preferred solvents, catalysts and reaction conditions are set forth in the appended illustrative Examples.

The invention further includes any variant of the present processes, in which an intermediate product obtainable at any stage thereof is used as starting material and the remaining steps are carried out, or in which the reaction components are used in the form of their salts.

Compounds of the invention and intermediates can also be converted into each other according to methods generally known per se.

The present invention also relates to any novel starting materials, intermediates and processes for their manufacture.

Depending on the choice of starting materials and methods, the new compounds may be in the form of one of the possible isomers or mixtures thereof, for example, as substantially pure geometric (cis or trans) isomers, diastereomers, optical isomers, racemates or mixtures thereof. The aforesaid possible isomers or mixtures thereof are within the purview of the present invention.

Any resulting mixtures of isomers can be separated on the basis of the physicochemical differences of the constituents, into the pure geometric or optical isomers, diastereomers, for example, by fractional crystallization and/or chromatography, e.g., by high pressure liquid chromatography (HPLC) using a chiral adsorbent.

Finally, compounds of the invention are either obtained in the free form, or in a salt form thereof, preferably, in a pharmaceutically acceptable salt form thereof.

In particular, compounds of the invention which contain basic groups may be converted into acid addition salts, especially pharmaceutically acceptable acid addition salts. These are formed, e.g., with inorganic acids, such as mineral acids, e.g., sulfuric acid, phosphoric or hydrohalic acid, or with organic carboxylic acids, such as (C₁-C₄)-alkanecarboxylic acids which, e.g., are unsubstituted or substituted by halogen, e.g., acetic acid, such as saturated or unsaturated dicarboxylic acids, e.g., oxalic, succinic, maleic or fumaric acid, such as hydroxycarboxylic acids, e.g., glycolic, lactic, malic, tartaric or citric acid, such as amino acids, e.g., aspartic or glutamic acid, or with organic sulfonic acids, such as (C₁-C₄)-alkylsulfonic acids, e.g., methanesulfonic acid; or arylsulfonic acids which are unsubstituted or substituted (for example by halogen). Preferred are salts formed with hydrochloric acid, maleic acid and methanesulfonic acid. Salts may be formed using conventional methods, advantageously in the presence of an ethereal or alcoholic solvent, such as a lower alkohol. From the solutions of the latter, the salts may be precipitated with ethers, e.g., with diethyl ether or petroleum ether. Resulting salts may be converted into the free compounds by treatment with a suitable base, e.g., sodium hydroxide. These or other salts can also be used for the purification of the compounds obtained.

In view of the close relationship between the free compounds and the compounds in the form of their salts, whenever a compound is referred to a corresponding salt is also intended, provided such is possible or appropriate under the circumstances.

The compounds, including their salts, can also be obtained in the form of their hydrates, or include other solvents used for their crystallization.

As described herein above, the compounds of the present invention are allosteric enhancers of the A₁ adenosine receptor. Thus, the present invention provides a method for the modulation of the A₁ adenosine receptor in mammals which method comprises administering to a mammal in need thereof a therapeutically effective amount of a compound of formula (I).

Furthermore, compounds of formula (I) may be employed for the treatment of conditions mediated by the A₁ adenosine receptor. Such compounds may, thus, be employed therapeutically for the treatment of pain, in particular, chronic pain such as neuropathic pain, and inflammatory pain, cardiac disease or disorder such as cardiac disarrhythmias, e.g., peroxysmal supraventricular tachycardia, angina, myocardial infarction and stroke, neurological disease or injury, sleep disorders, epilepsy and depression.

In other words, the present invention provides a method for the treatment of conditions mediated by the A₁ adenosine receptor, which comprises administering to a mammal in need thereof a therapeutically effective amount of a compound of the present invention.

As used throughout the specification and in the claims, the term “treatment” embraces all the different forms or modes of treatment as known to those of the pertinent art and in particular includes preventive, curative, delay of progression and palliative treatment.

The term “therapeutically effective amount” as used herein refers to an amount of a drug or a therapeutic agent that will elicit the desired biological or medical response of a tissue, system or an animal (including man) that is being sought by a researcher or clinician.

The term “mammal” or “patient” are used interchangeably herein and include, but are not limited to, humans, dogs, cats, horses, pigs, cows, monkeys, rabbits, mice and laboratory animals. The preferred mammals are humans.

Preferably, the methods of the present invention are directed to the treatment of pain, including pain management generally, and particularly treatment and management of chronic pain, especially neuropathic pain. Neuropathic pain has been recognized as pain resulting from some type of pathological damage to or condition relating to the nervous system. Various types of neuropathic pain may be treated in accordance with the present invention, e.g., diabetic neuropathy and post herpetic neuralgia. Additional pathological conditions that can give rise to neuropathic pain that may be treated in accordance with the present invention include trigeminal neuralgia, AIDS associated neuropathies due to HIV infection and/or treatment, pain associated with cancer treatment, whip-lash pain, phantom limb pain, traumatic injury pain, complex regional pain syndrome, and pain due to peripheral vascular disease. Furthermore, methods of the present invention will be useful for the management and treatment of inflammatory and post surgical pain.

Preferred methods of the invention also include treatment of cardiac disease or disorder, and ischemia induced injuries, e.g., cardiac disarrhythmias, angina, myocardial infarction, stroke, and the like. Typical subjects for such treatments include, e.g., myocardial infarction, stroke, brain or spinal injury patients, patients undergoing major surgery such as heart surgery where brain ischemia is a potential complication, and the like.

Likewise, the present invention provides a method as defined above comprising co-administration, e.g., concomitantly or in sequence, of a therapeutically effective amount of a compound of formula (I), or a pharmaceutically acceptable salt thereof, and a second drug substance, said second drug substance being a hypolipidemic agent, an anti-inflammatory agent, an anti-hypertensive agent or an opioid analgesic agent, e.g., as indicated herein below.

The present invention further provides pharmaceutical compositions comprising a therapeutically effective amount of a compound of the present invention, alone or in combination with one or more pharmaceutically acceptable carriers.

In carrying out the methods of the present invention, the allosteric adenosine A₁ receptor enhancers of the present invention may be formulated into pharmaceutical compositions suitable for administration via a variety of routes, e.g., enteral such as oral or rectal, transdermal, intrathecal and parenteral administration to mammals, including man, for the treatment of conditions mediated by the A₁ adenosine receptor. Such conditions include, but are not limited to, pain, in particular, chronic pain such as neuropathic pain, and inflammatory pain, cardiac disease or disorder such as cardiac disarrhythmias, e.g., peroxysmal supraventricular tachycardia, angina, myocardial infarction and stroke, neurological disease or injury, sleep disorders, epilepsy and depression.

For oral administration the pharmaceutical composition comprising an allosteric adenosine A₁ receptor enhancer, or a pharmaceutically acceptable salt thereof, can take the form of solutions, suspensions, tablets, pills, capsules, powders, microemulsions, unit dose packets and the like.

Thus, the compounds of the present invention may be employed in the manufacture of pharmaceutical compositions comprising a therapeutically effective amount thereof in conjunction or admixture with excipients or carriers suitable for administration via a variety of routes, in particular, for enteral or parenteral application. Preferred are tablets and hard or soft shell gelatin capsules comprising the active ingredient together with:
a) diluents, e.g., lactose, dextrose, sucrose, mannitol, sorbitol, cellulose, glycine and/or vegetable oils;
b) lubricants, e.g., silica, talcum, stearic acid, its magnesium or calcium salt and/or polyethyleneglycol; for tablets also
c) binders, e.g., magnesium aluminum silicate, starch paste, gelatin, tragacanth, methylcellulose, sodium carboxymethylcellulose and or polyvinylpyrrolidone; and if desired
d) disintegrants, e.g., starches, agar, alginic acid or its sodium salt, or effervescent mixtures; and/or
e) absorbents, colorants, flavors and sweeteners.

Injectable compositions are preferably aqueous isotonic solutions or suspensions, and suppositories are advantageously prepared from fatty emulsions or suspensions.

Said compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. Said compositions are prepared according to conventional mixing, granulating or coating methods, respectively, and contain about 0.1-75%, preferably about 1-50%, of the active ingredient.

Suitable formulations for transdermal application include a therapeutically effective amount of a compound of the invention with carrier. Advantageous carriers include absorbable pharmacologically acceptable solvents to assist passage through the skin of the host. Characteristically, transdermal devices are in the form of a bandage comprising a backing member, a reservoir containing the compound optionally with carriers, optionally a rate controlling barrier to deliver the compound of the skin of the host at a controlled and predetermined rate over a prolonged period of time, and means to secure the device to the skin.

A unit dosage for a mammal of about 50-70 kg may contain between about 0.005 mg and 2000 mg, advantageously between about 1-1000 mg of the active ingredient. The therapeutically effective dosage of active compound is dependent on the species of warm-blooded animal (mammal), the body weight, age and individual condition, on the form of administration, and on the compound involved.

Accordingly, the present invention provides pharmaceutical compositions as described above for the treatment of conditions mediated by the A₁ adenosine receptor including pain, in particular, chronic pain such as neuropathic pain, and inflammatory pain, cardiac disease or disorder such as cardiac disarrhythmias, e.g., peroxysmal supraventricular tachycardia, angina, myocardial infarction and stroke, neurological disease or injury, sleep disorders, epilepsy and depression.

The pharmaceutical compositions may contain a therapeutically effective amount of a compound of the invention as defined above, either alone or in a combination with another therapeutic agent, e.g., each at an effective therapeutic dose as reported in the art. Such therapeutic agents include:
a) hypolipidemic agents such as HMG-CoA (3-hydroxy-3-methyl-glutaryl coenzyme A) reductase inhibitors, squalene synthase inhibitors, FXR (farnesoid X receptor) and LXR (liver X receptor) ligands, cholestyramine, fibrates, nicotinic acid and aspirin;
b) anti-inflammatory agents;
c) anti-hypertensive agents, e.g., loop diuretics, ACE (angiotensin converting enzyme) inhibitors, inhibitors of the Na-K-ATPase membrane pump, NEP (neutral endopeptidase) inhibitors, ACE/NEP inhibitors, angiotensin II antagonists, renin inhibitors, β-adrenergic receptor blockers, inotropic agents, calcium channel blockers, aldosterone receptor antagonists, and aldosterone synthase inhibitors; and
d) opioid analgesic agents.

As described above, a compound of the present invention may be administered either simultaneously, before or after the other active ingredient, either separately by the same or different route of administration or together in the same pharmaceutical formulation.

The structure of the therapeutic agents known by their generic or trade names may be taken, e.g., from the actual edition of the standard compendium “The Merck Index” or from databases, e.g., Patents International (e.g. IMS World Publications). Any person skilled in the art is fully enabled to identify the active agents and, based on these references, likewise enabled to manufacture and test the pharmaceutical indications and properties in standard test models, both in vitro and in vivo.

Accordingly, the present invention provides pharmaceutical compositions comprising a therapeutically effective amount of a compound of the invention in combination with another therapeutic agent, preferably selected from hypolipidemic agents, anti-inflammatory agents, anti-hypertensive agents and opioid analgesic agents.

Since the present invention has an aspect that relates to treatment with a combination of compounds which may be co-administered separately, the invention also relates to combining separate pharmaceutical compositions in kit form. The kit comprises two separate pharmaceutical compositions: (1) a composition comprising a compound of formula (I), or a pharmaceutically acceptable salt thereof, plus a pharmaceutically acceptable carrier or diluent; and (2) a composition comprising a hypolipidemic agent, an anti-inflammatory agent, an anti-hypertensive agent, or an opioid analgesic agent, or a pharmaceutically acceptable salt thereof, plus a pharmaceutically acceptable carrier or diluent. The amounts of (1) and (2) are such that, when co-administered separately, a beneficial therapeutic effect(s) is achieved. The kit comprises a container for containing the separate compositions such as a divided bottle or a divided foil packet, wherein each compartment contains a plurality of dosage forms (e.g., tablets) comprising (1) or (2). Alternatively, rather than separating the active ingredient-containing dosage forms, the kit may contain separate compartments each of which contains a whole dosage which in turn comprises separate dosage forms. An example of this type of kit is a blister pack wherein each individual blister contains two (or more) tablets, one (or more) tablet(s) comprising a pharmaceutical composition (1), and the second (or more) tablet(s) comprising a pharmaceutical composition (2). Typically the kit comprises directions for the administration of the separate components. The kit form is particularly advantageous when the separate components are preferably administered in different dosage forms (e.g., oral and parenteral), are administered at different dosage intervals, or when titration of the individual components of the combination is desired by the prescribing physician. In the case of the present invention a kit therefore comprises:
(1) a therapeutically effective amount of a composition comprising a compound of formula (I), or a pharmaceutically acceptable salt thereof, and a pharmaceutically acceptable carrier or diluent, in a first dosage form;
(2) a composition comprising a hypolipidemic agent, an anti-inflammatory agent, an anti-hypertensive agent, or an opioid analgesic agent, or a pharmaceutically acceptable salt thereof, in an amount such that, following administration, a beneficial therapeutic effect(s) is achieved, and a pharmaceutically acceptable carrier or diluent, in a second dosage form; and
(3) a container for containing said first and second dosage forms.

The present invention further relates to pharmaceutical compositions as described above for use as a medicament.

The present invention further relates to use of pharmaceutical compositions or combinations as described above for the preparation of a medicament for the treatment of conditions mediated by the A₁ adenosine receptor including pain, in particular, chronic pain such as neuropathic pain, and inflammatory pain, cardiac disease or disorder such as cardiac disarrhythmias, e.g., peroxysmal supraventricular tachycardia, angina, myocardial infarction and stroke, neurological disease or injury, sleep disorders, epilepsy and depression.

Thus, the present invention also relates to a compound of formula (I) for use as a medicament, to the use of a compound of formula (I) for the preparation of a pharmaceutical composition for the treatment of conditions mediated by the A₁ adenosine receptor, and to a pharmaceutical composition for use in conditions mediated by the A₁ adenosine receptor comprising a compound of formula (I), or a pharmaceutically acceptable salt thereof, in association with a pharmaceutically acceptable diluent or carrier therefore.

Finally, the present invention provides a method or use which comprises administering a compound of formula (I) in combination with a therapeutically effective amount of a hypolipidemic agent, an anti-inflammatory agent, an anti-hypertensive agent or an opioid analgesic agent.

Ultimately, the present invention provides a method or use which comprises administering a compound of formula (I) in the form of a pharmaceutical composition as described herein.

The above-cited properties are demonstrable in vitro and in vivo tests using advantageously mammals, e.g., mice, rats, dogs, monkeys or isolated organs, tissues and preparations thereof. Said compounds can be applied in vitro in the form of solutions, e.g., preferably aqueous solutions, and in vivo either enterally, parenterally, advantageously intrathecal or intravenously, e.g., as a suspension or in aqueous solution. The dosage in vitro may range between about 10⁻² molar and 10⁻¹⁰ molar concentrations. A therapeutically effective amount in vivo may range depending on the route of administration, between about 0.000001 mg/kg and 1000 mg/kg, preferably between about 0.00001 mg/kg and 100 mg/kg, more preferably between about 0.001 mg/kg and 10 mg/kg.

The activity of compounds according to the invention may be assessed using methods well-described in the art, e.g., as described herein below:

Membrane Preparation from CHO Cells Transfected with the Human Recombinant A₁, A_{2A} and A₃ Adenosine Receptors

The hCHO-A₁, hCHO-A_{2A} and hCHO-A₃ cell clones are grown adherently and maintained in Dulbecco's modified Eagle's medium with nutrient mixture F12, containing 10% fetal calf serum, penicillin (100 U/mL), streptomycin (100 μg/mL), L-glutamine (2 mM), geneticine (G418) 0.2mg/mL at 37° C. in 5% CO₂/95% air. 30 min at 37° C. (Klotz et al. Naunyn-Schmied. Arch Pharm. 1998, 357, 1-9). Cells are split two or three times weekly at a ratio of between 1:5 and 1:10. For membrane preparation the culture medium is removed. The cells are washed with PBS and scraped off T75 flasks in ice-cold hypotonic buffer (5 mM Tris HCl, 2 mM EDTA, pH 7.4). The cell suspension is homogenized with Polytron and the homogenate is spun for 10 min at 1,000×g. The supernatant is then centrifuged for 30 min at 100,000×g. The membrane pellet is resuspended in 50 mM Tris HCl buffer pH 7.4 for The A1 adenosine receptors, 50 mM Tris HCl buffer pH 7.4, 10 mM MgCl₂ for A_{2A} adenosine receptors, 50 mM Tris HCl buffer pH 7.4, 10 mM MgCl₂, 1 mM EDTA for A₃ adenosine receptors and incubated with 3 Ul/mL of adenosine deaminase for 30 min at 37° C. The protein concentration is determined according to a Bio-Rad method (Bradford, 1976) with bovine albumin as a standard reference.

### Adenosine Receptor Binding Experiments

To determine the effect of the compounds of the present invention on the binding to A₁, A_{2A} and A₃ receptors, membranes from hCHO-A₁, hCHO-A_{2A}, hCHO-A₃ are incubated in a buffer solution in the absence and in the presence of the examined compounds. Test agents are dissolved in DMSO and added to the assay from a 100-fold concentrated solution in DMSO. Control incubations also contain 1% DMSO. Bound and free radioactivity are separated by filtering the assay mixture through Whatman GF/B glass fibre filters using a Micro-mate 196 cell harvester (Packard Instrument Company). The filter bound radioactivity is counted on Top Count Microplate Scintillation Counter (efficacy 57%) with Micro-Scint 20.

Saturation Binding of [³H]CCPA to hCHO-A₁

Saturation binding experiments of [³H]CCPA (0.05 to 20 nM) to human A₁ receptors expressed in CHO membranes are performed in triplicate at 25° C. for 1 h in 50 mM Tris-HCl, pH 7.4, in the absence and presence of the tested compounds (10 μM). Non specific binding is defined as binding in the presence of 1 μM R-PIA.

Competition Binding of [³H]CCPA to hCHO-A₁

Competition experiments are carried out in triplicate in a final volume of 250 μL in test tubes containing 1 nM [³H]CCPA, 50 mM Tris-HCl, pH 7.4 and 100 μL of diluted membranes and at least six to eight different concentrations of the tested compounds in the range from 1 nM to 50 μM for 90 min at 25° C. (Baraldi et al. J. Med. Chem. 2003, 46, 794-809). Non specific binding is defined as binding in the presence of 1 μM R-PIA. Allosteric enhancement is measured as the action of different concentrations of the tested compounds to increase the specific binding of 1 nM [³H]CCPA to hCHO-A₁ membranes.

### Competition Binding Experiments

Competition experiments of 1 nM [³H]DPCPX (Borea et al. Life Sciences 1996, 59, 1373-1388), 2 nM [³H]ZM 241385 (Borea et al. Biochem. Pharmacol. 1995, 49, 461-469) and 2 nM [³H]MRE 3008F20 (Varani et al. Mol. Pharmacol 2000, 57, 968-975) to hCHO-A₁, hCHO-A_{2A} and hCHO-A₃ are performed incubating membranes (100 μg of protein/assay) at 25° C. for 90 min, at 4° C. for 60 min and at 4° C. for 150 min, respectively. Competition experiments are performed in duplicate in a final volume of 100 μL in test tubes containing 50 mM Tris HCl buffer (10 mM MgCl₂, 1 mM EDTA for A₃), pH 7.4 and 100 μL of membranes and at least six to eight different concentrations of the test compound. Non-specific binding is defined as the binding in the presence of 1 μM DPCPX, ZM 241385 and MRE 3008F20 for A₁, A_{2A} and A₃, respectively, and is about 30% of total binding.

[³H]DPCPX (specific activity, 120 Ci/mmol) and [³H]CCPA (specific activity, 55 Ci/mmol) may be obtained from NEN Research Products (Boston, Mass.); [³H]ZM 241385 (specific activity, 17 Ci/mmol) may be obtained from Tocris Cookson (Bristol, UK); [³H]MRE 3008F20 (specific activity, 67 Ci/mmol) may be obtained from Amersham International (Buckinghamshire, UK).

Measurement of cAMP Content in CHO Cells (Functional Assay)

Allosteric enhancement is measured as the ability of a test compound at different concentrations (0.01, 0.1, 1 and 10 μM) to reduce the cAMP content of hCHO-A₁ cells. To initiate an experiment, growth medium is removed from the 12-well plates and cells are washed once with warm Hanks' buffered saline. The wash solution is then removed and replaced with fresh Hanks' solution containing forskolin (1 μM), rolipram (20 μM), N⁶- cyclopentyladenosine (CPA, 0.01 nM), adenosine deaminase (2 U/mL), and the test compound. Forskolin is used to stimulate the activity 15 of adenylyl cyclase, rolipram to inhibit cAMP phosphodiesterase, adenosine deaminase to degrade endogenous adenosine, and CPA to cause a small increase of the number of activated adenosine receptors. After 6 min of incubation at 36° C. in the presence of a test compound, the incubation solution is removed and hydrochloric acid (final concentration 50 mM) is added to terminate drug action. The content of cAMP in 20 acidified extracts of cells is determined by radioimmunoassay as previously described (Kollias-Baker et al. J. Pharmacol. Exp. Ther. 1997, 281, 761-768). Because the magnitude of the effects of allosteric enhancers on hCHO-A₁ cells change subtly with passage number and differ slightly among different aliquots of cells, the actions of the test compounds and the action of a reference compound (PD 81,723) are assessed in 25 each experiment. The effect of each test compound on cAMP content is presented as a percentage of the value of cAMP content in the absence of drug (control, 100%).

### Chronic Inflammatory Pain Model

The intraplantar injection of zymosan-induced mechanical hyperalgesia may be used as a model of chronic inflammatory pain (Meller et al., Neuropharmacology, 33:1471-1478, 1994). In this model, typically male Sprague-Dawley or Wistar rats (200-250 g) receives an intraplantar injection of 3 mg/100 μL zymosan into one hind paw. A marked inflammation occurs in this hind paw. Drugs are generally administered for evaluation of efficacy, 24 h after the inflammatory insult, when mechanical hyperalgesia is considered fully established.

### Chronic Neuropathic Pain Models

Three animal models of chronic neuropathic pain may be used that involve some form of peripheral nerve damage. in the Seltzer model (Seltzer et al., Pain, 43: 205-218, 1990) Sprague-Dawley or Wistar rats (200-250 g) are anaesthetized and a small incision made mid-way up one thigh (usually the left) to expose the sciatic nerve. The nerve is carefully cleared of surrounding connective tissues at a site near the trochanter just distal to the point at which the posterior biceps semitendinosus nerve branches off the common sciatic nerve. A 7-0 silk suture is inserted into the nerve with a 3/8 curved, reversed-cutting mini-needle, and tightly ligated so that the dorsal 1/3 to 1/2 of the nerve thickness is held within the ligature. The muscle and skin are closed with sutures and clips and the wound dusted with antibiotic powder. In sham animals the sciatic nerve is exposed but not ligated and the wound closed as in nonsham animals.

In the Chronic Constriction Injury (CCI) model (Bennett, G. J. and Xie, Y. K. Pain, 33: 87-107, 1988) rats are anaesthetized and a small incision is made mid-way up one thigh (usually the left) to expose the sciatic nerve. The nerve is cleared of surrounding connective tissue and four ligatures of 4/0 chromic gut are tied loosely around the nerve with approximately 1 mm between each, so that the ligatures just barely constrict the surface of the nerve. The wound is closed with sutures and clips as described above. In sham animals the sciatic nerve is exposed but not ligated and the wound closed as in nonsham animals.

In contrast to the Seltzer and CCI models, the Chung model involves ligation of the spinal nerve (Kim, S. O. and Chung, J. M. Pain, 50: 355-363, 1992). In this model, Sprague-Dawley or Wistar rats (200-250 g) are anesthetized and placed into a prone position and an incision is made to the left of the spine at the L4-S2 level. A deep dissection through the paraspinal muscles and separation of the muscles from the spinal processes at the L4-S2 level will reveal part of the sciatic nerve as it branches to form the L4, L5 and L6 spinal nerves. The L6 transverse process is carefully removed with a small rongeur enabling visualization of these spinal nerves. The L5 spinal nerve is isolated and tightly ligated with 7-0 silk suture. The wound is closed with a single muscle suture (6-D silk) and one or two skin closure clips and dusted with antibiotic powder. In sham animals the L5 nerve is exposed as before but not ligated and the wound closed as before.

### Behavioral Index

In all chronic pain models (inflammatory and neuropathic) mechanical hyperalgesia is assessed by measuring paw withdrawal thresholds of both hind paws to an increasing pressure stimulus using an Analgesymeter. Mechanical allodynia is assessed by measuring withdrawal thresholds to non-noxious mechanical stimuli applied with von Frey hairs to the planter surface of both hind paws. Thermal hyperalgesia is assessed by measuring withdrawal latencies to a noxious thermal stimulus applied to the underside of each hind paw. With all models, mechanical hyperalgesia and allodynia and thermal hyperalgesia develop within 1-3 days following surgery and persist for at least 50 days. For the assays described herein, drugs may be applied before and after surgery to assess their effect on the development of hyperalgesia, approximately 14 days following surgery, to determine their ability to reverse established hyperalgesia.

The percentage reversal of hyperalgesia is calculated as follows:

$$\% reversal = \frac{postdose threshold - predose threshold}{naive threshold - prepose threshold} \times 100$$

In the above described pain models, all surgery may be performed under enflurane/O₂ inhalation anesthesia. In all cases the wound is closed after the procedure and the animals are allowed to recover. In all pain models employed, after a few days, in all but the sham operated animals, a marked mechanical and thermal hyperalgesia and allodynia develops in which there is a lowering of pain threshold and an enhanced reflex withdrawal response of the hind paw to touch, pressure or thermal stimuli. After surgery, the animals may also exhibit characteristic changes to the affected paw. In the majority of animals the toes of the affected hind paw are held together and the foot is turned slightly to one side, and in some rats the toes are also curled under. The gait of the ligated rats varies, but limping is uncommon. Some rats are seen to raise the affected hind paw from the cage floor and demonstrate an unusual rigid extension of the hind limb when held. The rats tend to be very sensitive to touch and may vocalize. Otherwise the general health and condition of the rats is good.

Illustrative of the invention, the compounds of Example 2, Example 16-9 and Example 30 increase the A₁ specific binding of the agonist [³H]CCPA to human CHO-A₁ membranes up to 4.4-fold, 5.5-fold and 4.2-fold, respectively, when tested at 10 μM concentration. Likewise, the compounds of Example 2, Example 16-9 and Example 30 exhibit about 3.5-fold, 6.3-fold and 4.6-fold increase in the B_{MAX} value of the agonist [³H]CCPA, respectively, when tested at 10 μM concentration.

The following Examples are intended to illustrate the invention and are not to be construed as being limitations thereon. If not mentioned otherwise, all evaporations are performed under reduced pressure, preferably between about 10 mmHg and 100 mmHg. The structure of final products, intermediates and starting materials is confirmed by standard analytical methods, e.g., microanalysis, melting point (m.p.) and spectroscopic characteristics, e.g., MS, IR and NMR. Abbreviations used are those conventional in the art.

### EXAMPLE 1

### {(2-Amino-4-[(diisopropylamino)methyl]-5-phenylthiophen-3-yl}(4-chlorophenyl)methanone, Method D

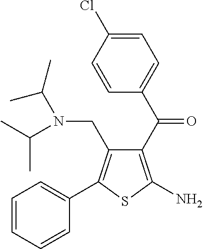

### A. (2-Amino-4-methylthiophen-3-yl)(4-chlorophenyl)methanone

To a suspension of 3-(4-chlorophenyl)-3-oxo-propionitrile (900 mg, 5 mmol) and 2,5-dimethyl-[1,4]dithiane-2,5-diol (450 mg, 2.5 mmol) in absolute EtOH (10 mL), cooled in a bath of water/ice (4° C.), is added TEA (5 mmol, 0.7 mL). After stirring for 10 min at RT, the mixture is refluxed for 2 h. The resulting red-brown solution is cooled and concentrated, and the residue dissolved in EtOAc (10 mL). The organic phase is subsequently washed with 1% w/v aqueous HCl (5 mL), a saturated solution of NaHCO₃ (5 mL), water (5 mL) and brine (5 mL), dried (Na₂SO₄) and concentrated to give a brown residue. The residue is suspended in ethyl ether (15 mL), the suspension stirred for 30 min and filtered. The filtrate is concentrated, suspended with petroleum ether and the resulting suspension is stirred for 30 min and filtered. The filtrate is concentrated, and the residue is purified by column chromatography using a mixture of EtOAc:petroleum ether—2:8 as eluent to give (2-amino-4-methylthiophen-3-yl)(4-chlorophenyl)methanone as an orange solid, m.p.: 148-150° C. ¹H NMR (CDCl₃): δ 1.66 (s, 3H), 5.85 (s, 1H), 6.61 (br s, 2H), 7.38 (d, J=6.4 Hz, 2H), 7.45 (d, J=6.4 Hz, 2H); IR (KBr) cm⁻¹: 3345, 1589, 1435, 1267.

### B. 2-[3-(4-Chlorobenzoyl)-4-methylthiophen-2-yl]isoindole-1,3-dione

The title A compound (755 mg, 3 mmol) is dissolved in acetic acid (20 mL), then to the solution is added phthalic anhydride (3.6 mmol, 533 mg) and the mixture is heated under reflux for 15 h. The solvent is evaporated and the residual material is dissolved in EtOAc (20 mL). The organic solution is washed with a saturated solution of NaHCO₃ (5 mL), water (5 mL) and brine (5 mL), dried (Na₂SO₄) and concentrated. The residue is stirred for 1 h with petroleum ether (20 mL), and the solids are collected by filtration to afford 2-[3-(4-chlorobenzoyl)-4-methylthiophen-2-yl]isoindole-1,3-dione as a brown powder, ¹H NMR (CDCl₃): δ 2.24 (s, 3H), 7.02 (s, 1H), 7.22 (d, J=7.2 Hz, 2H), 7.62-8.00 (m, 6H).

### C. 2[5-Bromo-3-(4-chlorobenzoyl)-4-methylthiophen-2-yl]isoindole-1,3-dione

To a solution of the title B compound (20 mmol, 7.6 g) in benzene (150 mL) is added benzoyl peroxide (484 mg, 2 mmol) and the mixture is heated under reflux. At refluxing conditions, a mixture of NBS (20 mmol, 3.56 g) and benzoyl peroxide (484 mg, 2 mmol) is added and the mixture is refluxed for 6 h further. The solvent is removed under reduced pressure, and the residue is dissolved in EtOAc (330 mL). The organic solution is subsequently washed a saturated solution of NaHCO₃ (200 mL), water (50 mL) and brine (50 mL), dried (Na₂SO₄) and concentrated to give a brown powder. The powder is suspended with petroleum ether (200 mL), the mixture is stirred for 30 min and the solids are collected by filtration to afford 2-[5-bromo-3-(4-chlorobenzoyl)-4-methylthiophen-2-yl]isoindole-1,3-dione which is used as such in the next step without further purification, m.p.: 194-195° C. ¹H NMR (CDCl₃): δ 2.09 (s, 3H), 7.19 (d, J=7.4 Hz, 2H), 7.62-7.71 (m, 6H); IR (KBr) cm⁻¹: 1728, 1664, 1587, 1368, 717.

### D. 2-[3-(4-Chlorobenzoyl)-4-methyl-5-phenylthiophen-2-yl]isoindole-1,3-dione

The title D compound is prepared from the title C compound following the procedure described by Romagnoli et al. in J. Med. Chem. 2006, 49(13), 3906-3915 (General procedure D). The product is purified by column chromatography (eluent EtOAc:petroleum ether—1.5:8.5 as eluent) to afford 2-[3-(4-chlorobenzoyl)-4-methyl-5-phenylthiophen-2-yl]isoindole-1,3-dione as a brown solid, m.p.: 223-225° C. ¹H NMR (CDCl₃): δ 2.24 (s, 3H), 7.24 (d, J=8.4 Hz, 2H), 7.74 (m, 5H), 7.80 (m, 6H).

### E. 2-[4-Bromomethyl-3-(4-chlorobenzoyl)-5-phenylthiophen-2-yl]isoindole-1,3-dione

To a refluxing suspension of the title A compound (458 mg, 1 mmol) in CCl₄ (10 mL), is added NBS (180 mg, 1 mmol) and benzoyl peroxide (14 mg, 0.06 mmol) and the mixture is refluxed for 1 h. After this time, a mixture of N-bromosuccinimide (180 mg, 1 mmol.) and benzoyl peroxide (14 mg, 0.06 mmo) is added and the mixture refluxed for another hour. The yellow solution is then cooled to RT, and succinimide that separates upon cooling is removed by filtration and the filtercake is washed with CCl₄ (5 mL). The filtrate is washed with 5% NaHCO₃ solution (5 mL), water (5 mL), brine (50 mL), dried over Na₂SO₄, and concentrated to give a yellow solid which is suspended with petroleum ether (10 mL). The mixture is stirred for 30 min, and the solid is collected by filtration to afford 2-[4-bromomethyl-3-(4-chlorobenzoyl)-5-phenylthiophen-2-yl]isoindole-1,3-dione which is used as such for the next reaction without further purification, m.p.: 160-161° C. ¹H NMR (CDCl₃): δ 4.73 (s, 2H), 7.21 (d, J=8.6 Hz, 2H), 7.48 (d, J=8.6 Hz, 2H), 7.52 (m, 1H), 7.68 (m, 8H).

### F. 2-{3-(4-Chlorobenzoyl)-4-[(diisopropylamino)methyl]-5-phenylthiophen-2-yl}isoindole-1,3-dione

To a stirred solution of the title E compound (265 mg, 0.5 mmol) in dry DMF (5 mL) is added K₂CO₃ (70 mg, 0.5 mmol). The mixture is cooled with a bath of ice/water, and then diisopropylamine (4 equiv, 2 mmol) is added. The mixture is stirred at RT for 2 h. After this time, the solvent is removed under reduced pressure, and the residue is taken up in a mixture of EtOAc (15 mL) and water (5 mL). The organic phase is washed with brine (5 mL), dried over Na₂SO₄, and concentrated under vacuo to give a brown residue which is purified by column chromatography (EtOAc:petroleum ether - 3:7 as eluent) to afford 2-{3-(4-chlorobenzoyl)-4-[(diisopropylamino)methyl]-5-phenylthiophen-2-yl}isoindole-1,3-dione as a white solid, m.p.: 168-170° C. ¹H NMR (CDCl₃): δ 0.57 (d, J=6.6 Hz, 12H), 2.62 (m, 2H), 3.71 (s, 2H), 7.22 (d, J=8.6 Hz, 2H), 7.47 (m, 2H), 7.74 (m, 9H).

### G. {2-Amino-4-[(diisopropylamino)methyl]-5-phenylthiophen-3-yl}(4-chlorophenyl)methanone

A stirred suspension of the title F compound (0.5 mmol) and 100% hydrazine monohydrate (1.2 eq, 0.6 mmol, 29 μL) in absolute EtOH (10 mL) is refluxed for 1 h. After this time, the solvent is evaporated and the residue is portioned between EtOAc (10 mL) and water (5 mL). The separated organic phase is washed with brine (2 mL), dried, and concentrated under vacuo to obtain a residue which is purified by column chromatography (EtOAc:petroleum ether—7:3 as eluent) to give {2-amino-4-[(diisopropylamino)methyl]-5-phenylthiophen-3-yl}(4-chlorophenyl)methanone as a yellow solid, m.p.: 185-187° C. ¹H NMR (CDCl₃): δ 0.53 (d, J=6.6 Hz, 12H), 2.39 (m, 2H), 3.43 (s, 2H), 5.33 (br s, 2H), 7.39 (d, J=8.2 Hz, 2H), 7.80 (d, J=8.2 Hz, 2H).

### EXAMPLE 2

### {2-Amino-4-[(dicyclohexylamino)methyl]-5-phenyithiophen-3-yl}(4-chlorophenyl)methanone, Method D

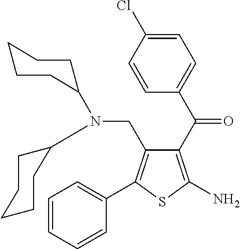

The title compound is prepared analogously as described in Example 1, and purified by column chromatography (EtOAc:petroleum ether—2:8 as eluent). Yellow solid, m.p.: 201-203° C. ¹H NMR (CDCl₃): δ 0.88 (m, 8H), 1.16 (m, 6H), 1.51 (m, 6H), 1.74 (m, 2H), 3.51 (s, 2H), 5.41 (br s, 2H), 7.36 (m, 5H), 7.41 (d, J=8.6 Hz, 2H), 7.78 (d, J=8.6 Hz, 2H).

### EXAMPLE 3

### {2-Amino-4-[(diethylamino)methyl]-5-phenylthiophen-3-yl}(4-chlorophenyl)methanone, Method D

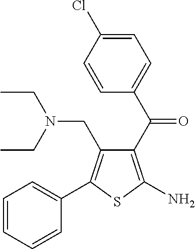

The title compound is prepared analogously as described in Example 1, and purified by column chromatography (EtOAc:petroleum ether—7:3 as eluent). Yellow solid, m.p.: 176-178° C. ¹H NMR (CDCl₃): δ 0.54 (d, J=7.2 Hz, 6H), 2.02 (d, J=7.2 Hz, 4H), 3.24 (s, 2H), 5.89 (br s, 2H), 7.36 (d, J=8.8 Hz, 2H), 7.41 (d, J=8.8 Hz, 2H), 7.55 (m, 4H).

### EXAMPLE 4

### {2-Amino-4-[(diallylamino)methyl]-5-phenylthiophen-3-yl}(4-chlorophenyl)methanone, Method D

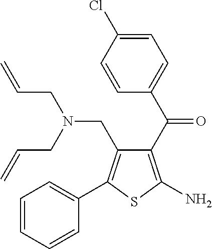

The title compound is prepared analogously as described in Example 1, and purified by column chromatography (EtOAc:petroleum ether—1.5:8.5 as eluent). Yellow solid, m.p.: 155-156° C. ¹H NMR (CDCl₃): δ 2.42 (d, J=6.0 Hz, 4H), 3.21 (s, 2H), 4.85 (m, 4H), 5.22 (m, 2H), 5.72 (br s, 2H), 7.39 (m, 7H), 7.70 (d, J=8.4 Hz, 2H).

### EXAMPLE 5

### {2-Amino-4-[(dipropylamino)methyl]-5-phenylth iophen-3-yl}(4-chlorophenyl)methanone, Method D

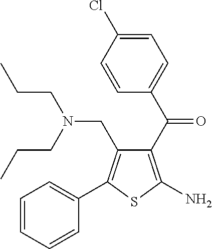

The title compound is prepared analogously as described in Example 1, and purified by column chromatography (EtOAc:petroleum ether—3:7 as eluent). Yellow oil, ¹H NMR (CDCl₃): δ 0.48 (t, J=7.2 Hz, 6H), 0.99 (m, 4H), 1.73 (t, J=7.2 Hz, 4H), 3.21 (s, 2H), 5.84 (br s, 2H), 7.34 (m, 5H), 7.43 (d, J=7.8 Hz, 2H), 7.67 (d, J=7.6 Hz, 2H).

### EXAMPLE 6

### {2-Amino-4-[(t-butyl(methyl)amino)methyl]-5-phenylthiophen-3-yl}(4-chlorophenyl)methanone, Method D

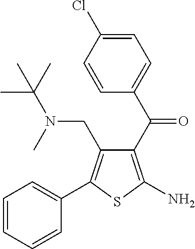

The title compound is prepared analogously as described in Example 1, and purified by column chromatography (EtOAc:petroleum ether—6:4 as eluent). Yellow solid, m.p.: 220-222° C. ¹H NMR (CDCl₃): δ 0.54 (s, 9H), 1.63 (m, 3H), 3.34 (s, 2H), 5.30 (br s, 2H), 7.37 (m, 7H), 7.75 (d, J=8.4 Hz, 2H).

### EXAMPLE 7

### {2-Amino-4-[(benzyl(methyl)amino)methyl]-5-phenylthiophen-3-yl}(4-chlorophenyl)methanone, Method D

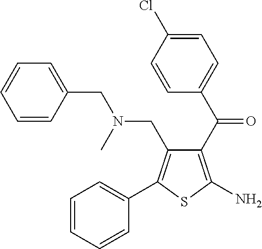

The title compound is prepared analogously as described in Example 1, and purified by column chromatography (EtOAc:petroleum ether—4:6 as eluent). Yellow solid, m.p.: 86-88° C. ¹H NMR (CDCl₃): δ 1.74 (s, 3H), 3.23 (m, 2H), 4.34 (m, 2H), 5.78 (br s, 2H), 7.19 (m, 2H), 7.25 (m, 7H), 7.47 (m, 5H).

### EXAMPLE 8

### {2-Amino-4-[(dimethylamino)methyl]-5-phenylthiophen-3-yl}(4-chlorophenyl)methanone, Method D

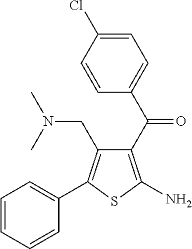

The title compound is prepared analogously as described in Example 1, and purified by column chromatography (EtOAc as eluent). Yellow oil, ¹H NMR (CDCl₃): δ 1.58 (s, 6H), 2.96 (s, 2H), 5.77 (br s, 2H), 7.35 (m, 7H), 7.61 (d, J=8.4 Hz, 2H).

### EXAMPLE 9

### {2-Amino-4-[(butyl(methyl)amino)methyl]-5-phenylthiophen-3-yl}(4-chlorophenyl)methanone, Method D

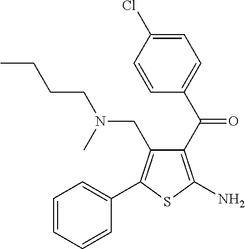

The title compound is prepared analogously as described in Example 1, and purified by column chromatography (EtOAc:petroleum ether—7:3 as eluent). Yellow solid, m.p.: 84-88° C. ¹H NMR (CDCl₃): δ 0.78 (t, J=7.2 Hz, 3H), 0.89 (m, 4H), 1.82 (m, 3H), 2.03 (t, J=7.2 Hz, 2H), 3.75 (s, 2H), 5.68 (br s, 2H), 7.29 (m, 3H), 7.39 (m 2H), 7.49 (m, 4H).

### EXAMPLE 10

### {2-Amino-5-(4-cyanophenyl)-4-[(dicyclohexylamino)methyl]thiophen-3-yl}(4-chlorophenyl)methanone, Method D

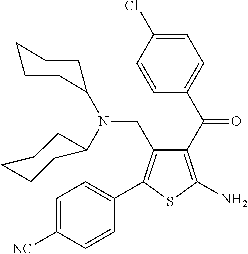

### A. 2-[3-(4-Chlorobenzoyl)-5-(4-cyanophenyl)-4-methylthiophene-2-yl]isoindole-1,3-dione

A stirred solution of title C compound of Example 1 (0.691 g, 1.5 mmol) and 4-cyanophenyl-boronic acid (0.33 g, 2.25 mmol) in 1,4-dioxane (15 mL containing 2 drops of water) is degassed under a stream of nitrogen over 10 min, then treated with [1,1′-bis(diphenylphosphino)-ferrocene]dichloropalladium(II) methylene chloride complex (123 mg, 0.15 mmol) and cesium fluoride (0.57 g, 3.75 mmol). The reaction mixture is heated under nitrogen at 45° C. for 30 min, then at 65° C. for 3 h. The reaction mixture is concentrated in vacuo, taken up in DCM, and loaded onto a short column of silica gel and eluted with 5% EtOAc in DCM to afford 2-[3-(4-chlorobenzoyl)-5-(4-cyanophenyl)-4-methylthiophene-2-yl]isoindole-1,3-dione as a pale yellow solid. This material is used without further purification.

### B. 2[4-Bromomethyl-3-(4-chlorobenzoyl)-5-(4-cyanophenyl)thiophene-2-yl]isoindole-1,3-dione

The title A compound is dissolved in 1,2-dichloroethane (10 mL), treated with NBS (0.32 g, 1.8 mmol), and heated to reflux under nitrogen with stirring. Benzoyl peroxide 75% (50 mg, 0.155 mmol) is added, and heating at reflux is continued. After 1.5 h (reaction incomplete by TLC), additional NBS (0.178 g, 1.0 mmol) is added, followed by 75% benzoyl peroxide (26 mg, 0.08 mmol), and heating at reflux is continued for 1.5 h further. The mixture is cooled to RT and added directly to a column of silica gel. The column is eluted with DCM, then with 2% EtOAc in DCM to afford 2-[4-bromomethyl-3-(4-chlorobenzoyl)-5-(4-cyanophenyl)thiophene-2-yl]isoindoline-1,3-dione as a pale tan solid, m.p.: 211-214° C. MS: 584.9 (M+Na). ¹H-NMR (CDCl₃): δ 7.83 (m, 2H), 7.79 (m, 2H), 7.71-7.76 (m, 4H), 7.69 (d, 2H, J=8.5 Hz), 7.21 (d, 2H, J=8.5 Hz), 4.68 (s, 2H).

### C. 2-{3-(4-Chlorobenzoyl)-5-(4-cyanophenyl)-4-[(dicyclohexylamino)methyl}thiophen-2-yl]isoindole-1,3-dione

A stirred solution/suspension of the title B compound (281 mg, 0.50 mmol) in a 2:1-mixture of ACN and THF (6 mL) under nitrogen is treated with dicyclohexylamine (0.40 mL, 2 mmol), then heated at 60° C. for 2 h. The mixture is concentrated and the solvents replaced with DCM (25 mL). Aqueous NaOH (0.1 N, 6 mL) is added and the mixture is stirred for a few minutes, then separated. The organic solution is washed with water (2×15 mL) and brine (15 mL), dried (Na₂SO₄), and filtered. The filtrate is applied directly to a column of silica gel and the column eluted with 2% EtOAc in DCM, then with 3:1-mixture of heptane and EtOAc to afford 2-{3-(4-chlorobenzoyl)-5-(4-cyanophenyl)-4-[(dicyclohexylamino)methyl]thiophen-2-yl}isoindole-1,3-dione as a light purple solid. This material is used without further purification.

### D. {2-Amino-5-(4-cyanophenyl)-4-[(dicyclohexylamino)methyl]thiophen-3-yl}(4-chlorophenyl)methanone

The title C compound is dissolved in EtOH (3 mL), cooled on an ice bath, and treated dropwise with a solution of ethylenediamine (120 mg, 2 mmol) in EtOH (1 mL). The stirred mixture is allowed to warm to RT over 15 min, maintained at RT for 30 min, heated at reflux for 1 h, then cooled to RT, and diluted with DCM (20 mL). The organic solution is washed with water (10 mL), brine (10 mL), dried (Na₂SO₄), and concentrated in vacuo. The residue is dissolved in minimum DCM and the solution loaded onto a silica gel column and eluted with 3:1-mixture of heptane and EtOAc to afford crude {2-amino-5-(4-cyanophenyl)-4-[(dicyclohexylamino)methyl]-thiophen-3-yl}(4-chlorophenyl)methanone as a yellow solid which is trituration with ACN and dried in vacuo afforded of the final product as a yellow solid, m.p.: 196-200° C. MS: 532.2 (M+H). ¹H NMR (CDCl₃): δ 7.76 (d, 2H, J=8.5 Hz), 7.66 (d, 2H, J=8.5 Hz), 7.47 (d, 2H, J=8.5 Hz), 7.44 (d, 2H, J=8.5 Hz), 5.48 (br s, 2H), 3.53 (s, 2H), 1.77 (m, 2H), 1.40-1.55 (m, 6H), 1.10-1.20 (m, 4H), 0.65-0.95 (m, 10H).

### EXAMPLE 11

### {2-Amino-5-(4-t-butylphenyl)-4-[(dicyclohexylamino)methyl]thiophen-3-yl}(4-chlorophenyl)methanone, Method D

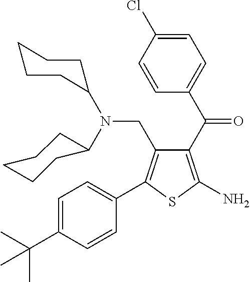

The title compound is prepared analogously as described in Example 10. m.p.: 180-184° C. MS: 563.2 (M+H). ¹H NMR (CDCl₃): δ 7.78 (d, 2H, J=8.5Hz), 7.42 (d, 2H, J=8.5 Hz), 7.38 (d, 2H, J=8.5 Hz), 7.28 (d, 2H, J=8.5 Hz), 5.35 (br s, 2H), 3.51 (s, 2H), 1.77 (m, 2H), 1.40-1.54 (m, 6H), 1.11-1.19 (m, 4H), 0.67-0.97 (m, 10H).

### EXAMPLE 12

### {2-Amino-5-(4-chlorophenyl)-4-[(dicyclohexylamino)methyl]thiophen-3-yl}(4-chlorophenyl)methanone, Method D

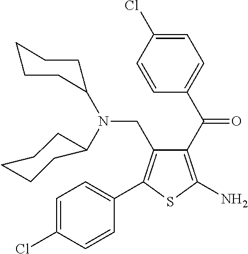

The title compound is prepared analogously as described in Example 10. m.p.: 200-204° C. MS: 541.2 (M+H). ¹H NMR (CDCl₃): δ 7.76 (d, 2H, J=8 Hz), 7.42 (d, 2H, J=8 Hz), 7.35 (d, 2H, J=8.5 Hz), 7.29 (d, 2H, J=8.5 Hz), 5.43 (s, 2H), 3.48 (s, 2H), 1.76 (m, 2H), 1.40-1.55 (m, 6H), 1.10-1.20 (m, 4H), 0.65-0.95 (m, 10H).

### EXAMPLE 13

### {2-Amino-4-[(dicyclohexylamino)methyl]-5-(2-fluorophenyl)thiophen-3-yl}(4-chlorophenyl)methanone, Method D

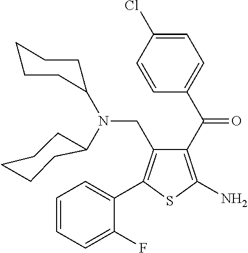

The title compound is prepared analogously as described in Example 10. m.p.: 223-225° C. MS: 525.2 (M+H). ¹H NMR (CDCl₃): δ 7.76 (d, 2H, J=8.5 Hz), 7.42 (d, 2H, J=8.5 Hz), 7.29-7.36 (m, 2H), 7.09-7.19 (m, 2H), 5.46 (br s, 2H), 3.47 (s, 2H), 1.77 (m, 2H), 1.47-1.55 (m, 4H), 1.39-1.47 (m, 2H), 1.10-1.20 (m, 4H), 0.67-0.98 (m, 10H).

### EXAMPLE 14

### {2-Amino-4-[(dicyclohexylamino)methyl]-5-(3-fluorophenyl)thiophen-3-yl}(4-chlorophenyl)methanone, Method D

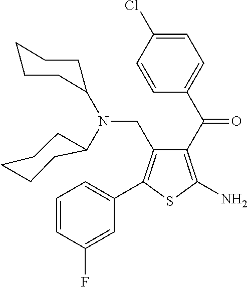

The title compound is prepared analogously as described in Example 10. m.p.: 205-207° C. MS: 525.2 (M+H). ¹H NMR (CDCl₃): δ 7.77 (d, 2H, J=8.5 Hz), 7.43 (d, 2H, J=8.5 Hz), 7.34 (m, 1 H), 7.14 (m, 1H), 7.09 (m, 1H), 7.01 (m, 1H), 5.40 (br s, 2H), 3.52 (s, 2H), 1.77 (m, 2H), 1.48-1.55 (m, 4H), 1.40-1.48 (m, 2H), 1.13-1.20 (m, 4H), 0.68-0.98 (m, 10H).

### EXAMPLE 15

### {2-Amino-4-[(dicyclohexylamino)methyl]-5-(4-fluorophenyl)thiophen-3-yl}(4-chlorophenyl)methanone, Method D

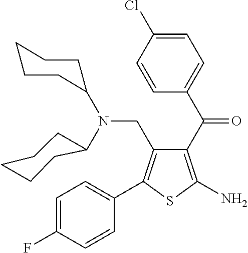

The title compound is prepared analogously as described in Example 10. m.p.: 222-224° C. MS: 525.2 (M+H). ¹H NMR (CDCl₃): δ 7.77 (d, 2H, J=8.5 Hz), 7.42 (d, 2H, J=8.5 Hz), 7.33 (dd, 2H, J=9.0, 5.5 Hz), 7.07 (t, 2H, J=9.0 Hz), 5.37 (br s, 2H), 3.47 (s, 2H), 1.76 (m, 2H), 1.47-1.55 (m, 4H), 1.40-1.47 (m, 2H), 1.11-1.19 (m, 4H), 0.67-0.97 (m, 10H).

### EXAMPLE 16

The following compounds may be prepared analogously as described in Examples 1 and 10 (Method D).

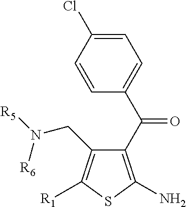

| Example | | | | Melting | Molecular |
|---|---|---|---|---|---|
| No. | R₁ | R₅ | R₆ | point, (° C.) | ion, (M + H) |
| 16-1 | Phenyl | n-Butyl | n-Butyl | Oil | 455.2 |
| 16-2 | Phenyl | i-Butyl | i-Butyl | 127-133 | 455.2 |
| 16-3 | Phenyl | n-Pentyl | n-Pentyl | Oil | 483.2 |
| 16-4 | Phenyl | Cyclopentyl | Cyclopentyl | 135-141 | 479.2 |
| 16-5 | Phenyl | Cyclohexyl | n-Propyl | 162-165 | 467.2 |
| 16-6 | Phenyl | Cyclohexyl | Allyl | 151-157 | 465.2 |
| 16-7 | Phenyl | 2-Methoxy-2-oxoethyl | Methyl | 156.5-160 | 429.0 |
| 16-8 | Phenyl | 2-Methoxyethyl | 2-Methoxyethyl | 140-145 | 459.0 |
| 16-9 | Phenyl | Neopentyl | Neopentyl | Oil | 483.2 |
| 16-10 | Phenyl | 1-Adamantyl | H | 135-140 | 477.2 |
| 16-11 | Phenyl | 3,4-Dichlorophenyl | Methyl | 80-84 | 501.0 |
| 16-12 | Phenyl | 4-Methoxyphenyl | Methyl | 131.5-136 | 463.0 |
| 16-13 | Phenyl | 4-Methylphenyl | Methyl | 173-176 | 447.0 |
| 16-14 | 3,4-(Dichloro)- | Cyclohexyl | Cyclohexyl | 222-225 | 577.0 |
| | phenyl | | | | |
| 16-15 | 4-(i-Propoxy)- | Cyclohexyl | Cyclohexyl | 175-177 | 565.2 |
| | phenyl | | | | |

EXAMPLE 17

### {2-Amino-4-[(dicyclohexylamino)methyl]-5-(3,5-difluorophenyl)thiophen-3-yl}(4-chlorophenyl)methanone, Method B

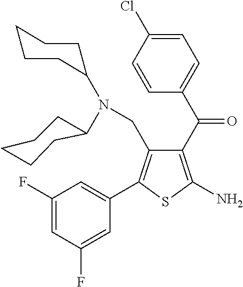

### A. 2-[5-Bromo-4-bromomethyl-3-(4-chlorobenzoyl)hiophen-2-yl]isoindole-1,3-dione

A stirred solution of the title C compound of Example 1 (9.16 g, 20 mmol) and NBS (3.92 g, 22 mmol) in 1,2-dichloroethane (100 mL) is heated at 80° C. under nitrogen, then treated with dibenzoylperoxide (75%, 0.65 g, 2 mmol), stirred for 1.5 h, and then cooled to RT. Heptane (150 mL) is added, the suspension is stirred for a few minutes, and filtered. The filter cake is rinsed with a 2:1-mixture of heptane and DCM, the combined filtrates are loaded directly onto a silica gel column, and eluted with a 2:1-mixture of heptane and DCM to afford 2-[5-bromo-4-bromomethyl-3-(4-chlorobenzoyl)thiophene-2-yl]isoindole-1,3-dione as a yellow solid, m.p. 173-175° C. ¹H NMR (CDCl₃) δ 4.65 (s, 2H), 7.20 (d, J=6.6 Hz, 2H), 7.66 (d, J=6.6 Hz, 2H), 7.62-7.71 (m, 4H). IR (KBr) cm⁻¹: 1727, 1658, 1348, 1330, 1084.

### B. 2-{5-Bromo-3-(4-chlorobenzoyl)-4-[(dicyclohexylamino)methyl]thiophen-2-yl}isoindole-1,3-dione

The title A compound (7.12g, 13.2 mmol) is dissolved in a 2:1-mixture of ACN and THF (150 mL), cooled on ice, and treated dropwise with dicyclohexylamine (9.3 mL, 46.5 mmol). The stirred solution is heated at 60° C. for 4 h, during which a solid formed, and then cooled to RT, and concentrated in vacuo. The residue is taken up in DCM (200 mL), cooled on ice, treated with 0.25 N NaOH (60 mL), stirred for a few minutes, and partitioned. The organic solution is washed with water (2×100 mL), brine (100 mL), dried (Na₂SO₄), filtered, and concentrated in vacuo. The residual solid is triturated with heptane and dried in vacuo to afford 2-{5-bromo-3-(4-chlorobenzoyl)-4-[(dicyclohexylamino)methyl]thiophen-2-yl}isoindole-1,3-dione as a pale tan solid (stored in freezer), m.p. 173-5° C. MS: 641.0 (M+H). ¹H NMR (CDCl₃): δ 7.79 (m, 2H), 7.71 (m, 2H), 7.67 (d, 2H, J=8.5 Hz), 7.24 (d, 2H, J=8.5 Hz), 3.81 (s, 2H), 2.10-2.20 (m, 2H), 1.60-1.70 (m, 4H), 1.49-1.57 (m, 2H), 1.34-1.43 (m, 4H), 1.00-1.15 (m, 10H).

### C. {2-Amino-4-[(dicyclohexylamino)methyl]-5-(3,5-difluorophenyl)thiophen-3-yl}(4-chlorophenyl)methanone

A stirred mixture of the title B compound (0.45 g, 0.70 mmol) and 3,5-difluorophenylboronic acid (0.166 g, 1.05 mmol) in toluene (7 mL) is degassed under a stream of nitrogen over 10 min, then treated with [1,1′-bis(diphenylphosphino)ferrocene]dichloropalladium(II) methylene chloride complex (57 mg, 0.07 mmol) and cesium fluoride (0.266 g, 1.75 mmol), heated at 55° C. for 45 min, then at 75° C. for 20 h. After cooling to RT, the reaction mixture is diluted with DCM (20 mL) and filtered through Celite®. The filtrate is concentrated in vacuo and the residue taken up in EtOH (10 mL) containing hydrazine hydrate (50 mg, 1.0 mmol), heated at reflux for 3 h, cooled and stirred at RT for 3 h further. The solution is concentrated in vacuo, and the residue dissolved in minimal amount of DCM, then loaded onto a silica gel column, and eluted with a 4:1-mixture of heptane and EtOAc to afford a yellow solid, which is triturated with ACN to afford {2-amino-4-[(dicyclohexylamino)methyl]-5-(3,5-difluorophenyl)thiophen-3-yl}(4-chlorophenyl)methanone as a yellow solid, m.p. 163-166° C. MS: 543.2 (M+H). ¹H NMR (CDCl₃): δ 7.75 (d, 2H, J=8.5 Hz), 7.43 (d, 2H, J=8.5 Hz), 6.91 (m, 2H), 6.76 (m, 1 H), 5.42 (br s, 2H), 3.52 (s, 2H), 1.79 (m, 2H), 1.49-1.58 (m, 4H), 1.42-1.49 (m, 2H), 1.15-1.23 (m, 4H), 0.70-1.00 (m, 10H).

### EXAMPLE 18

### {2-Amino-4-[(dicyclohexylamino)methyl]-5-(4-methylphenyl)thiophen-3-yl}(4-chlorophenyl)methanone, Method B

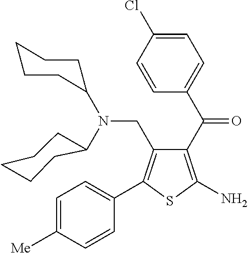

The title compound is prepared analogously as described in Example 17. m.p.: 191-194° C. MS: 521.2 (M+H). ¹H NMR (CDCl₃): δ 7.78 (d, 2H, J=8.5 Hz), 7.42 (d, 2H, J=8.5 Hz), 7.24 (d, 2H, J=8 Hz), 7.17 (d, 2H, J=8 Hz), 5.39 (br s, 2H), 3.50 (s, 2H), 2.38 (s, 3H), 1.74 (m, 2H), 1.46-1.54 (m, 4H), 1.39-1.46 (m, 2H), 1.11-1.20 (m, 4H), 0.65-0.97 (m, 10H).

### EXAMPLE 19

### {2-Amino-4-[(dicyclohexylamino)methyl]-5-(4-methoxyphenyl)thiophen-3-yl}(4-chlorophenyl)methanone, Method B

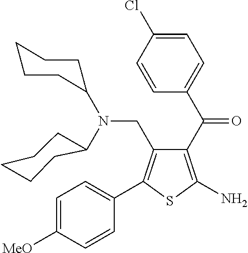

The title compound is prepared analogously as described in Example 17. m.p.: 178-182° C. MS: 537.2 (M+H). ¹H NMR (CDCl₃): δ 7.78 (d, 2H, J=8.5 Hz), 7.42 (d, 2H, J=8.5 Hz), 7.28 (d, 2H, J=8 Hz), 6.91 (d, 2H, J=8 Hz), 5.38 (br s, 2H), 3.84 (s, 3H), 3.47 (s, 2H), 1.75 (m, 2H), 1.47-1.55 (m, 4H), 1.40-1.47 (m, 2H), 1.11-1.20 (m, 4H), 0.66-0.98 (m, 10H).

### EXAMPLE 20

### {2-Amino-4-[(diethylamino)methyl]thiophen-3-yl}(4-chlorophenyl)methanone, Method A

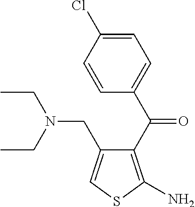

### A. (2-Amino-4-methylthiophen-3-yl)(4-chlorophenyl)methanone

To a suspension of 3-(4-chlorophenyl)-3-oxo-propionitrile (900 mg, 5 mmol) and 2,5-dimethyl-[1,4]dithiane-2,5-diol (450 mg, 2.5 mmol) in absolute EtOH (10 mL), cooled in a bath of water/ice (4° C.), is added TEA (5 mmol, 0.7 mL). After stirring for 10 min at RT, the mixture is refluxed for 2 h. The resulting red-brown solution is cooled and concentrated, and the residue dissolved in EtOAc (10 mL). The organic phase is subsequently washed with 1% w/v aqueous HCl (5 mL), a saturated solution of NaHCO₃ (5 mL), water (5 mL) and brine (5 mL), dried (Na₂SO₄) and concentrated to give a brown residue. The residue is suspended in ethyl ether (15 mL), the suspension stirred for 30 min and filtered. The filtrate is concentrated, suspended with petroleum ether and the resulting suspension is stirred for 30 min and filtered. The filtrate is concentrated, and the residue is purified by column chromatography using a mixture of EtOAc:petroleum ether—2:8 as eluent to give (2-amino-4-methylthiophen-3-yl)(4-chlorophenyl)-methanone as an orange solid, m.p.: 148-150° C. ¹H NMR (CDCl₃): δ 1.66 (s, 3H), 5.85 (s, 1H), 6.61 (br s, 2H), 7.38 (d, J=6.4 Hz, 2H), 7.45 (d, J=6.4 Hz, 2H); IR (KBr) cm⁻¹: 3345, 1589, 1435, 1267.

### B. 2-[3-(4-Chlorobenzoyl)-4-methylthiophen-2-yl]isoindole-1,3-dione

The title A compound (755 mg, 3 mmol) is dissolved in acetic acid (20 mL), then to the solution is added phthalic anhydride (3.6 mmol, 533 mg) and the mixture is heated under reflux for 15 h. The solvent is evaporated and the residual material is dissolved in EtOAc (20 mL). The organic solution is washed with a saturated solution of NaHCO₃ (5 mL), water (5 mL) and brine (5 mL), dried (Na₂SO₄) and concentrated. The residue is stirred for 1 h with petroleum ether (20 mL), and the solids are collected by filtration to afford 2-[3-(4-chlorobenzoyl)-4-methylthiophen-2-yl]isoindole-1,3-dione as a brown powder, ¹H NMR (CDCl₃): δ 2.24 (s, 3H), 7.02 (s, 1 H), 7.22 (d, J=7.2 Hz, 2H), 7.62-8.00 (m, 6H).

### C. 2-[5-Bromo-3-(4-chlorobenzoyl)-4-methylthiophen-2-yl]isoindole-1,3-dione

To a solution of the title B compound (20 mmol, 7.6 g) in benzene (150 mL) is added benzoyl peroxide (484 mg, 2 mmol) and the mixture is heated under reflux. At refluxing conditions, a mixture of NBS (20 mmol, 3.56 g) and benzoyl peroxide (484 mg, 2 mmol) is added and the mixture is refluxed for 6 h further. The solvent is removed under reduced pressure, and the residue is dissolved in EtOAc (330 mL). The organic solution is subsequently washed a saturated solution of NaHCO₃ (200 mL), water (50 mL) and brine (50 mL), dried (Na₂SO₄) and concentrated to give a brown powder. The powder is suspended with petroleum ether (200 mL), the mixture is stirred for 30 min and the solids are collected by filtration to afford 2-[5-bromo-3-(4-chlorobenzoyl)-4-methylthiophen-2-yl]isoindole-1,3-dione which is used as such in the next step without further purification, m.p.: 194-195° C. ¹H NMR (CDCl₃): δ 2.09 (s, 3H), 7.19 (d, J=7.4 Hz, 2H), 7.62-7.71 (m, 6H); IR (KBr) cm⁻¹: 1728, 1664, 1587, 1368, 717.

### D. 2-[5-Bromo-4-bromomethyl-3-(4-chlorobenzoyl)thiophen-2-yl]isoindole-1,3-dione

To a suspension of the title C compound (20 mmol, 9.2 g) in CCl₄ (150 mL) is added benzoyl peroxide (242 mg, 1 mmol) and the mixture is heated under reflux. At refluxing conditions, a mixture of NBS (20 mmol, 3.56 g) and benzoyl peroxide (242 mg, 1 mmol) is added and the mixture is refluxed for 1 h further. After this time, if the reaction is not finished, a mixture of NBS (2 mmol, 356 mg) and benzoyl peroxide (242 mg, 1 mmol) is added and the mixture is refluxed for another hour. The resulting yellow solution is cooled to RT and the precipitated succinimide is removed by filtration and washed with CCl₄ (25 mL). The filtrate is washed with 5% NaHCO₃ solution (50 mL), water (50 mL) and brine (50 mL), dried (Na₂SO₄) and concentrated to give a yellow powder. The powder is suspended with petroleum ether (100 mL), the mixture is stirred for 30 min and the solids are collected by filtration to give 2-[5-bromo-4-bromornethyl-3-(4-chlorobenzoyl)thiophen-2-yl]isoindole-1,3-dione as a yellow solid, m.p.: 173-175° C. ¹H NMR (CDCl₃): δ 4.65 (s, 2H), 7.20 (d, J=6.6 Hz, 2H), 7.66 (d, J=6.6 Hz, 2H), 7.62-7.71 (m, 4H). IR (KBr) cm⁻¹: 1727, 1658, 1348, 1330, 1084.

### E. 2-{5-Bromo-3-(4-chlorobenzoyl)-4-[(diethylamino)methyl]thiophen-2-yl}isoindole-1,3-dione

To a stirred solution of the title D compound (900 mg, 1.6 mmol) in dry DCM (5 mL) is added TEA (1.1 equiv, 1.76 mmol, 243 mg). The mixture is cooled with a bath of ice/water, and diethylamine (3 equiv, 5 mmol, 366 mg) is added. The mixture is stirred at RT for 2 h, diluted with DCM (5 mL), washed with water (5 mL) and brine (5 mL). The organic layer is dried (Na₂SO₄) and concentrated in vacuo, and the residue is purified by column chromatography to afford 2-{5-bromo-3-(4-chlorobenzoyl)-4-[(diethylamino)methyl]thiophen-2-yl}isoindole-1,3-dione.

### F. 2-{3-(4-Chlorobenzoyl)-4-[(diethylamino)methyl]thiophen-2-yl}isoindoline-1,3-dione

A solution of the title E compound (2 mmol) in DMF (20 mL), containing TEA (0.3 mL, 2 mmol, 1 equiv) is hydrogenated over 120 mg of 10% Pd/C at 60 psi for 3 h. The catalyst is removed by filtration, the filtrate is concentrated. The residue is dissolved in DCM (20 mL), washed with water (5 mL) and brine (5 mL), and dried (Na₂SO₄). The solvent is removed under reduced pressure and the residue is purified by column chromatography to afford 2-{3-(4-chloro-benzoyl)-4-[(diethylamino)methyl]thiophen-2-yl}isoindole-1,3-dione.

### G. {2-Amino-4-[(diethylamino)methyl]thiophen-3-yl}(4-chlorophenyl)methanone

A stirred suspension of the title F compound (0.5 mmol) and 100% hydrazine monohydrate (1.2 equiv, 0.6 mmol, 29 4) in absolute EtOH (10 mL) is heated at reflux for 3 h. After this time, the resulting solution is stirred at RT for 1 h further. The reaction is finished after the complete solubilization of the starting material. The solvent is evaporated and the residue is partitioned between EtOAc (10 mL) and water (5 mL). The organic phase is separated, washed with brine (2 mL), dried and concentrated in vacuo. The residue is purified by column chromatography to give {2-amino-4-[(diethylamino)methyl]thiophen-3-yl}(4-chlorophenyl)methanone as a yellow solid, m.p.: 78-80° C. ¹H NMR (CDCl₃): δ 1.26 (t, J=7.0 Hz, 6H), 2.17 (q, J=7.0 Hz, 4H), 3.08 (s, 2H), 5.99 (br s, 2H), 6.25 (s, 1 H), 7.36 (d, J=8.6 Hz, 2H), 7.52 (d, J=8.6 Hz, 2H).

### EXAMPLE 21

### {2-Amino-4-[(dipropylamino)methyl]thiophen-3-yl}(4-chlorophenyl)methanone, Method A

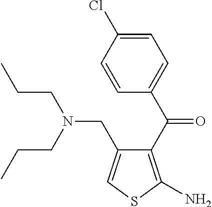

The title compound is prepared analogously as described in Example 20, and purified by column chromatography (EtOAc:petroleum ether—9.5:0.5 as eluent) to afford a yellow oil. ¹H NMR (CDCl₃): δ 0.69 (t, J=7.0 Hz, 6H), 1.19 (m, 4H), 1.96 (t, J=7.0 Hz, 4H), 3.01 (s, 2H), 6.07 (br s, 2H), 6.19 (s, 1H), 7.34 (d, J=8.4 Hz, 2H), 7.51 (d, J=8.4 Hz, 2H).

### EXAMPLE 22

### {2-Amino-4-[(diisopropylamino)methyl]thiophen-3-yl}(4-chlorophenyl)methanone, Method A

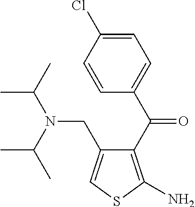

The title compound is prepared analogously as described in Example 20, and purified by column chromatography (EtOAc:petroleum ether—1:1 as eluent) to afford a brown oil. ¹H NMR (CDCl₃): δ 0.86 (d, J=6.6 Hz, 12H), 1.66 (m, 2H), 3.66 (s, 2H), 5.28 (s, 1H), 6.25 (br s, 2H), 7.38 (d, J=8.4 Hz, 2H), 7.52 (d, J=8.4 Hz, 2H).

### EXAMPLE 23

### {2-Amino-4-[(methyl(phenyl)amino)methyl]thiophen-3-yl}(4-chlorophenyl)methanone, Method A

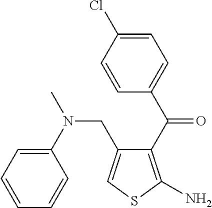

The title compound is prepared analogously as described in Example 20, and purified by column chromatography (EtOAc:petroleum—ether 2:8 as eluent) to afford a yellow solid. m.p.: 129-131° C. ¹H NMR (CDCl₃): δ 2.82 (s, 3H), 3.78 (s, 2H), 5.95 (s, 1H), 6.48 (br s, 2H), 6.67 (t, J=7.2 Hz, 1H), 7.12 (d, J=7.2 Hz, 2H), H), 7.18 (d, J=7.2 Hz, 2H), 7.38 (d, J=8.6 Hz, 2H), 7.49 (d, J=8.6 Hz, 2H).

### EXAMPLE 24

### {2-Amino-4-[(ethyl(phenyl)amino)methyl]thiophen-3-yl}(4-chlorophenyl)methanone, Method A

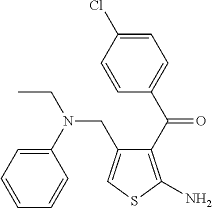

The title compound is prepared analogously as described in Example 20, and purified by column chromatography (EtOAc:petroleum ether—1.5:8.5 as eluent) to afford a yellow solid. m.p.: 130-132° C. ¹H NMR (CDCl₃): δ 1.02 (t, J=7.2 Hz, 3H), 3.26 (q, J=7_{—}2 Hz, 2H), 3,73 (s, 2H), 5.98 (s, 1H), 6.48 (m, 4H), 6.64 (t, J=7.2 Hz, 1H), 7.14 (t, J=7.6 Hz, 2H), 7.39 (d, J=8.4 Hz, 2H), 7.51 (d, J=8.4 Hz, 2H).

### EXAMPLE 25

### {2-Amino-4-[(4-fluorophenyl(methyl)amino)methyl]thiophen-3-yl}(4-chlorophenyl)methanone, Method A

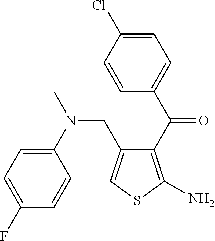

The title compound is prepared analogously as described in Example 20, and purified by column chromatography (EtOAc:petroleum ether—2:8 as eluent) to afford a yellow oil. ¹H NMR (CDCl₃): δ 2.74 (s, 3H), 3.75 (s, 2H), 5.97 (s, 1 H), 6.40 (m, 4H), 6.84 (t, J=8.4 Hz, 2H), 7.36 (d, J=8.8 Hz, 2H), 7.49 (d, J=8.6 Hz, 2H).

### EXAMPLE 26

### {2-Amino-4-[(4-chlorophenyl(methyl)amino)methyl]thiophen-3-yl}(4-chlorophenyl)methanone, Method A

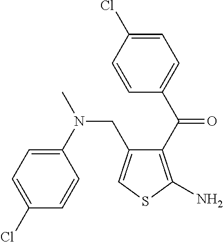

The title compound is prepared analogously as described in Example 20, and purified by column chromatography (EtOAc:petroleum ether—1.5:8.5 as eluent) to afford a yellow oil. ¹H NMR (CDCl₃): δ 2.79 (s, 3H), 3.77 (s, 2H), 5.91 (s, 1H), 6.41 (d, J=9.2 Hz, 2H), 6.46 (br s, 2H), 7.08 (d, J=9.2 Hz, 2H), 7.36 (d, J=8.8 Hz, 2H), 7.51 (d, J=8.8 Hz, 2H).

### EXAMPLE 27

### {2-Amino-4-[(methyl(4-trifluoromethylphenyl)amino)methyl]thiophen-3-yl}(4-chlorophenyl)methanone, Method A

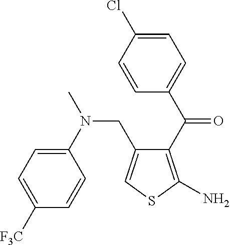

The title compound is prepared analogously as described in Example 20, and purified by column chromatography (EtOAc:petroleum ether—1.5:8.5 as eluent) to afford a yellow oil. ¹H NMR (CDCl₃): δ 2.88 (s, 3H), 3.85 (s, 2H), 5.87 (s, 1 H), 6.48 (m, 4H), 7.42 (m, 4H), 7.52 (d, J=8.8 Hz, 2H).

### EXAMPLE 28

### {2-Amino-4-[(benzyl(methyl)amino)methyl]-5-methylthiophen-3-yl}(4-chlorophenyl)methanone, Method C

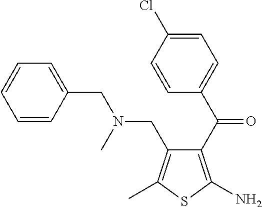

### A. 2-[3-(4-Chlorobenzoyl)-4,5-dimethylthiophen-2-yl]isoindole-1,3-dione

To a solution of (2-amino-4,5-dimethylthiophen-3-yl)(4-chlorophenyl)methanone (532 mg, 2 mmol; prepared as described in U.S. Pat. No. 6,323,214) in acetic acid (15 mL) is added phthalic anhydride (360 mg, 2.4 mmol) and the mixture is heated to reflux for 15 h. The solvent is evaporated in vacuo and the residual material is dissolved in EtOAc (20 mL). The organic solution is washed with a saturated aqueous solution of NaHCO₃ (5 mL), water (5 mL), then brine (5 mL), dried (Na₂SO₄), filtered, and concentrated in vacuo. The crude product is stirred for 1 h in petroleum ether (20 mL), then filtered, affording (2-[3-(4-chlorobenzoyl)-4,5-dimethylthiophen-2-yl]isoindole-1,3-dione as a yellow powder, ¹H NMR (CDCl₃): δ 2.10 (s, 3H), 2.43 (s, 3H), 7.24 (d, J=8.4 Hz, 2H), 7.64 (d, J=8.4 Hz, 2H), 7.78 (m, 4H).

### B. 2-[4-(Bromomethyl)-3-(4-chlorobenzoyl)-5-methylthiophen-2-yl]isoindole-1,3-dione

To the title A compound (2 mmol, 798 mg) in ACN (10 mL) is added NBS (2 mmol, 356 mg) and the mixture is heated at reflux for 2 h. After this time, another portion of NBS (2 mmol, 356 mg) is added and the reflux is continued for another 2 h. The solvent is then removed under reduced pressure, and the residue dissolved in DCM (15 mL), washed with water (5 mL), brine (5 mL), dried (Na₂SO₄), and concentrated to give a dark oil. This residue is then purified by flash chromathography (EtOAc:petroleum ether—2:8 as eluent) to furnish the compound as a yellow solid. The powder is suspended in petroleum ether (10 mL), the mixture is stirred for 30 min, and then filtered to give 2-[4-bromomethyl-3-(4-chlorobenzoyl)-5-methylthiophen-2-yl]isoindole-1,3-dione, m.p. 173-175° C. ¹H NMR (CDCl₃): δ 2.53 (s, 3H), 4.65 (s, 2H), 7.17 (d, J=8.4 Hz, 2H), 7.63 (d, J=8.4 Hz, 2H), 7.63 (m, 2H), 7.73 (m, 2H).

### C. 2-{3-(4-Chlorobenzoyl)-4-[(benzyl(methyl)amino)methyl]-5-methylthiophen-2-yl}isoindole-1,3-dione

To a stirred solution of the title B compound (900 mg, 0.5 mmol) in dry DMF (5 mL) is added K₂CO₃ (0.6 mmol, 83 mg). The mixture is cooled with a bath of ice/water, and then benzyl(methyl)amine (3 equiv, 1.5 mmol, 182 mg) is added. The mixture is stirred at room temperature for one hour, the solvent is then removed under reduced pressure, and a mixture of DCM (15 mL) and water (5 mL) is added to the residue. The organic phase is washed with brine (5 mL) and dried (Na₂SO₄), filtered, then concentrated in vacuo. The residue is purified by column chromatography to afford 2-{3-(4-chlorobenzoyl)-4-[(benzyl(methyl)amino)methyl]-5-methylthiophen-2-}isoindole-1,3-dione. Yellow solid, m.p.: 197-199° C. ¹H NMR (CDCl₃): δ 1.76 (s, 3H), 2.47 (s, 3H), 3.25 (s, 2H), 3.40 (s, 2H), 6.69 (m, 2H), 7.14 (m, 3H), 7.27 (m, 2H), 7.72 (m, 4H).

### D. {2-Amino-4-[(benzyl(methyl)amino)methyl-]-5-methyithiophen-3-yl}(4-chlorophenyl)methanone

A stirred suspension of the title C compound (0.5 mmol) and hydrazine monohydrate (0.6 mmol, 29 μL) in absolute EtOH (10 mL) is heated at reflux for 3 h. The resulting solution is cooled to RT and stirred for 1 h further. The reaction is finished after the complete solubilization of the starting material. The solvent is evaporated and the residue partitioned between DCM (10 mL) and water (5 mL). The separated organic phase is washed with brine (2 mL), dried (Na₂SO₄), filtered, and then concentrated in vacuo. The residue is purified by column chromatography to afford {2-amino-4-[(benzyl(methyl)amino)methyl]-5-methylthiophen-3-yl}(4-chlorophenyl)methanone. Yellow solid, m.p.: 114-116° C. ¹H NMR (CDCl₃): δ 1.47 (s, 3H), 2.23 (s, 3H), 2.98 (s, 2H), 3.00 (s, 2H), 5.81 (br s, 2H), 7.08 (d, J=6.8 Hz, 1 H), 7.24 (m, 2H), 7.33 (d, J=6.8 Hz, 2H), 7.55 (d, J=6.8 Hz, 2H).

### EXAMPLE 29

### {2-Amino-[(dicyclohexylamino)methyl]-5-(furan-3-yl)thiophen-3-yl}(4-chlorophenyl)methanone, Method B

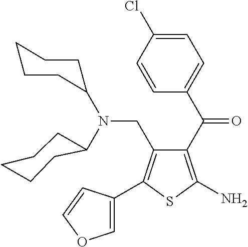

The title compound is analogously as described in Example 17. m.p.: 132-135° C. MS: 497.2 (M+H). ¹H NMR (CDCl₃): δ 7.74 (d, 2H, J=8.5 Hz), 7.50 (m, 1H), 7.45 (m, 1H), 7.41 (d, 2H, J=8.5 Hz), 6.47 (m, 1 H), 5.38 (br s, 2H), 3.50 (s, 2H), 1.78 (m, 2H), 1.50-1.58 (m, 4H), 1.40-1.50 (m, 2H), 1.20-1.28 (m, 4H), 0.73-0.98 (m, 10H).

### EXAMPLE 30

### {2-Amino-4-[(dicyclohexylamino)methyl]-5-(thiophen-2-yl)thiophen-3-yl}(4-chlorophenyl)methanone, Method B

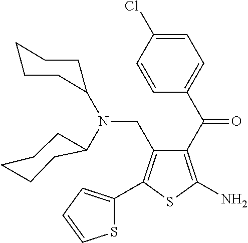

The title compound is prepared analogously as described in Example 17. m.p.: 197-199° C. MS: 513.2 (M+H). ¹H NMR (CDCl₃): δ 7.75 (d, 2H, J=8.5 Hz), 7.42 (d, 2H, J=8.5 Hz), 7.30 (m, 1H), 7.00-7.06 (m, 2H), 5.42 (br s, 2H), 3.62 (s, 2H), 1.77 (m, 2H), 1.49-1.57 (m, 4H), 1.40-1.48 (m, 2H), 1.19-1.28 (m, 4H), 0.72-0.99 (m, 10H).

### EXAMPLE 31

### {2-Amino-4-[(dicyclohexylamino)methyl]-5-(furan-2-yl)thiophen-3-yl}(4-chlorophenyl)methanone, Method B

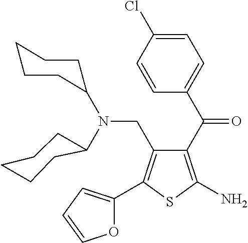

The title compound is prepared analogously as described in Example 17. m.p.: 158-160° C. MS: 497.2 (M+H). ¹H NMR (CDCl₃): δ 7.73 (d, 2H, J=8.5 Hz), 7.42 (m, 1H), 7.41 (d, 2H, J=8.5 Hz), 6.44 (m, 1 H), 6.35 (m, 1 H), 5.48 (br s, 2H), 3.62 (s, 2H), 1.79 (m, 2H), 1.50-1.58 (m, 4H), 1.40-1.48 (m, 2H), 1.24-1.31 (m, 4H), 0.75-1.00 (m, 10H).

### EXAMPLE 32

### {5-Amino-5′-chloro-3-[(dicyclohexylamino)methyl]-2,2′-bithiophen-4-yl}(4-chlorophenyl)methanone, Method D

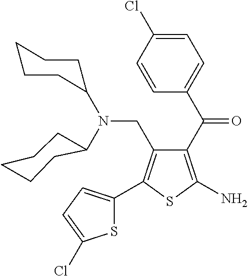

The title compound is prepared analogously as described in Example 17. m.p.: 195-197° C. MS (M+1) 547.1. ¹H NMR (CDCl₃): δ 7.73 (d, 2H, J=8.5 Hz), 7.42 (d, 2H, J=8.5 Hz), 6.85 (d, 1H, J=4 Hz), 6.78 (d, 1H, J=4 Hz), 5.42 (br s, 2H), 3.58 (s, 2H), 1.78 (m, 2H), 1.51-1.59 (m, 4H), 1.42-1.49 (m, 2H), 1.21-1.29 (m, 4H), 0.74-0.98 (m, 10H).

### EXAMPLE 33

### {2-Amino-4-[(dicyclohexylamino)methyl]-5-[4-(2-methoxyethoxy)phenyl]thiophen-3-yl}(4-chlorophenyl)methanone, Method E

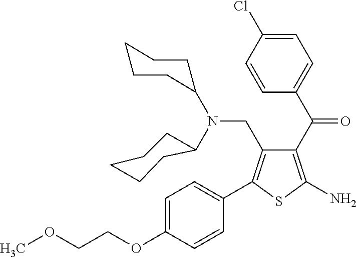

### A. 4-(Methoxyethoxy)-1-[(tri-n-butyl)stannyl]benzene

A stirred, cooled (−70° C.) solution of 4-(methoxyethoxy)-1-bromobenzene (1.155 g, 5.0 mmol) in anhydrous THF (12 mL) under nitrogen is treated dropwise with 2.4 N n-butyllithium in hexane (2.3 mL, 5.5 mmol) and stirred at −70° C. for 45 min. A solution of tri-n-butylstannyl chloride (1.82 mL, 6.7 mmol) is added dropwise so as to keep the pot temperature under -65° C., and the mixture is stirred at −70° C. for 2.5 h, then allowed to warm to RT, and the reaction is quenched with a 1:1-mixture of water and saturated ammonium chloride (10 mL). The mixture is extracted with EtOAc (50 mL) and the organic solution is separated, dried (MgSO₄), filtered, and concentrated in vacuo. The residual oil is dissolved in heptane and loaded onto a silica gel column and eluted with 0.1% of TEA in a 9:1-mixture of heptane and EtOAc to afford 4-(2-methoxyethoxy)-1-[(tri-n-butyl)stannyl]benzene as a colorless oil, MS: 465.2 (M+Na). ¹H NMR (CDCl₃): δ 7.35 (d, 2H, J=8.5 Hz), 6.92 (d, 2H, J=8.5 Hz), 4.12 (t, 2H, J=5 Hz), 3.75 (t, 2H, J=5 Hz), 3.45 (s, 3H), 1.51 (m, 6H), 1.32 (m, 6H), 1.02 (m, 6H), 0.88 (t, 9H, J=7.5 Hz).

### B. 2-{4-Bromomethyl-3-(4-chlorobenzoyl)-5-[4-(2-methoxyethoxy)phenyl]thiophene-2-yl}isoindol-t3-dione

A stirred solution of title C compound of Example 1 (0.461 g, 1.0 mmol) and the title A compound (0.552 g, 1.25 mmol) in 1,4-dioxane (10 mL) is degassed under a stream of nitrogen over 10 min, then treated with dichloropalladiumbis(triphenylphosphine) (70 mg, 0.1 mmol) and with lithium chloride (75 mg, 1.8 mmol) and heated under nitrogen at 100° C. for 1.5 h, then cooled to RT. The reaction mixture is diluted with DCM (40 mL), stirred a few minutes, filtered through Celite®, the filtrate concentrated in vacuo, and the residue dissolved in DCM, and loaded onto a short column of silica gel and eluted sequentially with DCM, then 1% of EtOAc in DCM, then 2% of EtOAc in DCM to afford 2-(3-(4-chlorobenzoyl)-5-(4-(methoxyethoxy)phenyl)-4-methylthiophene-2-yl)isoindole-1,3-dione as a yellow solid, which is used without further characterization. This intermediate is dissolved in 1,2-dichloroethane (7 mL), treated with NBS (0.267 g, 1.5 mmol), and heated to reflux under nitrogen with stirring. 75% Benzoyl peroxide (40 mg, 0.124 mmol) is added, and heated at reflux for 1.5 h further. More NBS (0.134 g, 0.75 mmol) and benzoyl peroxide (20 mg, 0.062 mmol) are added, and stirring is continued at reflux for 1 h more. The mixture is cooled to RT and added directly to a column of silica gel, and the column is eluted sequentially with 1% of EtOAc in DCM, then 2% of EtOAc in DCM, to afford 2-{4-bromomethyl-3-(4-chlorobenzoyl)-5-[4-(2-methoxyethoxy)phenyl]thiophene-2-yl}isoindole-1,3-dione as a pale yellow solid, m.p.: 142-145° C. MS 633.8 (M+Na). ¹H NMR (CDCl₃): δ 7.78 (m, 2H), 7.73 (m, 2H), 7.70 (d, 2H, J=8.5 Hz), 7.52 (d, 2H, J=8.5 Hz), 7.19 (d, 2H, J=8.5 Hz), 7.06 (d, 2H, J=8.5 Hz), 4.72 (s, 2H), 4.20 (t, 2H, J=5 Hz), 3.80 (t, 2H, J=5 Hz), 3.48 (s, 3H).

### C. {2-Amino-4-[(dicyclohexylamino)methyl]-5-[4-(2-methoxyethoxy)phenyl]thiophen-3-yl}(4-chlorophenyl)methanone

A stirred solution/suspension of the title B compound (305.5 mg, 0.50 mmol), in a 2:1-mixture of ACN and THF (6 mL) under nitrogen is treated with dicyclohexylamine (0.40 mL, 2 mmol), then heated at 60° C. for 2 h. The mixture is concentrated and the solvents are replaced with DCM (25 mL). Aqueous sodium hydroxide (0.1 N, 6 mL) is added, the mixture is stirred for a few minutes and the layers are separated. The organic solution is washed with water (2×15 mL), brine (15 mL), dried (Na₂SO₄), filtered, and concentrated in vacuo. The residue is dissolved in a 9:1-mixture of EtOH and water (3 mL), cooled in an ice bath, and treated dropwise with a solution of ethylenediamine (120 mg, 2 mmol) in EtOH (1 mL). The stirred mixture is allowed to warm to RT over 15 min, maintained at RT for 1 h, and diluted with DCM (20 mL). The solution is added to a pad of silica gel, eluted with EtOAc, and the filtrate concentrated in vacuo. The residue is dissolved in minimum DCM and the solution loaded onto a silica gel column and eluted with a 4:1-mixture of EtOAc and heptane to afford a yellow-orange solid, which is recrystallized from ACN to afford {2-amino-4-[(dicyclohexylamino)methyl]-5-[4-(2-methoxyethoxy)phenyl]thiophen-3-yl}(4-chlorophenyl)methanone as yellow-orange crystals, m.p.: 162-164° C. MS: 581.2 (M+H). ¹H NMR (CDCl₃): δ 7.77 (d, 2H, J=8.5 Hz), 7.41 (d, 2H, J=8.5 Hz), 7.27 (d, 2H, J=8.5 Hz), 6.93 (d, 2H, J=8.5 Hz), 5.38 (br s, 2H), 4.15 (t, 2H, J=5 Hz), 3.77 (t, 2H, J=5 Hz), 3.47 (br s, 5H), 1.74 (m, 2H), 1.47-1.54 (m, 4H), 1.40-1.46 (m, 2H), 1.11-1.20 (m, 4H), 0.66-0.96 (m, 10H).

### EXAMPLE 34

### {2-am ino-4-[(dicyclohexylamino)methyl]-5-(pyridin-2-yl)thiophen-3-yl}(4-chlorophenyl)methanone, Method E

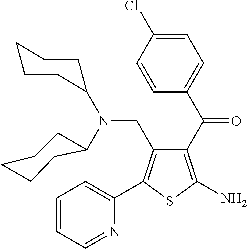

The title compound is prepared analogously as described in Example 35. m.p.: 192-194° C. MS: 508.2 (M+H). ¹H NMR (CDCl₃): δ 8.59 (m, 1H), 7.76 (d, 2H, J=8.5 Hz), 7.66 (m, 1H), 7.51 (d, 1H, J=8 Hz), 7.42 (d, 2H, J=8.5 Hz), 7.13 (m, 1H), 5.63 (br s, 2H), 3.73 (s, 2H), 1.84 (m, 2H), 1.48-1.56 (m, 4H), 1.39-1.47 (m, 2H), 1.20-1.28 (m, 4H), 0.74-0.98 (m, 10H).

### EXAMPLE 35

### {2-amino-4-[(dicyclohexylamino)methyl]-5-(pyridin-3-yl)thiophen-3-yl}(4-chlorophenyl)methanone, Method E

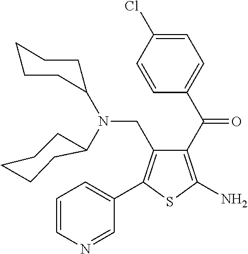

The title compound is prepared analogously as described in Example 35. m.p.: 168-170° C. MS: 508.2 (M+H). ¹H NMR (CDCl₃): δ 8.63 (m, 1H), 8.55 (m, 1H), 7.77 (d, 2H, J=8.5 Hz), 7.68 (m, 1 H), 7.44 (d, 2H, J=8.5 Hz), 7.32 (m, 1 H), 5.45 (br s, 2H), 3.51 (s, 2H), 1.78 (m, 2H), 1.48-1.55 (m, 4H), 1.40-1.47 (m, 2H), 1.10-1.18 (m, 4H), 0.66-0.98 (m, 10H).

### EXAMPLE 36

### {2-amino-4-[(dicyclohexylamino)methyl]-5-(pyridin-4-yl)thiophen-3-yl}(4-chlorophenyl)methanone, Method E

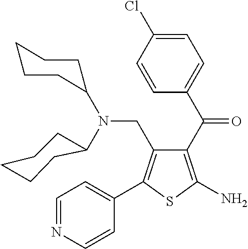

The title compound is prepared analogously as described in Example 35. m.p.: 214-216° C. MS: 508.2 (M+H). ¹H NMR (CDCl₃): δ 8.60 (d, 2H, J=6 Hz), 7.77 (d, 2H, J=8.5 Hz), 7.44 (d, 2H, J=8.5 Hz), 7.28 (d, 2H, J=6 Hz), 5.49 (br s, 2H), 3.58 (s, 2H), 1.77 (m, 2H), 1.48-1.55 (m, 4H), 1.41-1.47 (m, 2H), 1.13-1.21 (m, 4H), 0.68-0.98 (m, 10H).

### EXAMPLE 37

### (E)-{2-Amino-5-(3-cyclohexylprop-1-enyl)-4-[(dicyclohexylamino)methyl]thiophen-3-yl}(4-chlorophenyl)methanone, Method F

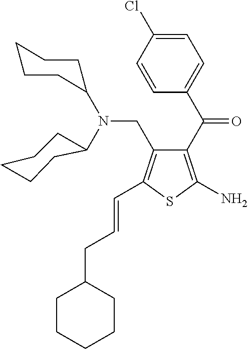

### A. 2-(5-Bromo-3-(4-chlorobenzoyl)-4-(hydroxymethyl)thiophen-2-yl)isoindole-1,3-dione

A stirred solution of the title A compound of Example 17 (8.09 a, 15 mmol) in THF (250 mL) is treated with 10% aqueous sodium bicarbonate (100 mL) and refluxed for 14 h, then cooled to RT and the layers are separated. The organic layer is concentrated in vacuo and the residue is dissolved in toluene (500 mL) and glacial acetic acid (5 mL), then refluxed for 19 h, treated with additional glacial acetic acid (5 mL), and refluxed for 5 h more. The mixture is concentrated in vacuo, the residue is dissolved in DCM, loaded onto a pad of silica gel, and eluted with a 1:1-mixture of EtOAc and heptane. The eluent containing the desired compound is concentrated, then dissolved in ACN, treated with powdered charcoal (3 g), warmed with stirring for a few minutes, and filtered through Celite®. The filtrate is concentrated in vacuo and the residual solid recrystallized from EtOAc/heptane (2 crops) to afford 2-(5-bromo-3-(4-chlorobenzoyl)-4-(hydroxymethyl)thiophene-2-yl)isoindole-1,3-dione as a very pale tan solid, m.p.: 170-172° C. MS: 499.8 (M+Na). ¹H NMR (CDCl₃): δ 7.71-7.78 (m, 4H), 7.59 (d, 2H, J=8.5 Hz), 7.16 (d, 2H, J=8.5 Hz), 4.61 (d, 2H, J=6Hz).

### B. (E)-2-[3-(4-Chlorobenzoyl)-5-(3-cyclohexylprop-1-enyl)-4-(hydroxymethyl)thiophen-2-yl]isoindole-1,3-dione

A stirred, cooled (3° C.) solution of 3-cyclohexyl-1-propyne (0.611 g, 5 mmol) in anhydrous THF (5 mL) under nitrogen is treated dropwise with 1 N catecholborane/THF (6 mL, 6 mmol), the mixture is refluxed for 2 h and concentrated in vacuo. The residue is cooled on ice and quenched with 1 N HCl (12 mL), then DCM (30 mL) is added, and the mixture is stirred for a few minutes and the layers are separated. The organic solution is washed with water (2×15 mL), dried (Na₂SO₄), and concentrated to approximately to 10 mL volume and diluted with 1,4-dioxane (10 mL). The remainder of the DCM is removed in vacuo, and the 1,4-dioxane solution is transferred to a 3-neck, 50 mL flask under nitrogen. To the flask is added the title A compound (477 mg, 1.0 mmol) and two drops of water, and the solution is degassed under a stream of nitrogen for 10 min. The mixture is treated with [1,1′-bis(diphenylphosphino)-ferrocene]dichloropalladium (II) methylene chloride complex (82 mg, 0.10 mmol) and cesium fluoride (0.50 g, 3.3 mmol), heated to 45° C. for 1 h with stirring, then to 70° C. for 1 h, and finally cooled to RT (reaction complete by LCMS analysis). DCM (30 mL) is added, and the mixture is filtered through Celite® (washing the cake with additional DCM). The filtrate is concentrated in vacuo, dissolved in a minimum of DCM, loaded onto a silica gel column, and eluted with a 3:1 mixture of heptane and EtOAc to afford a tan foam after concentration those fractions containing the desired product. The foam is triturated with petroleum ether containing a small amount of EtOAc to afford (E)-2-[3-(4-chlorobenzoyl)-5-(3-cyclohexylprop-1-enyl)-4-(hydroxymethyl)thiophen-2-yl]isoindole-1,3-dione as a pale tan solid, m.p.: 113-116° C. MS: 502.0 (M-OH). NMR (CDCl₃): δ 7.73 (m, 4H), 7.58 (d, 2H, J=8.5 Hz), 7.14 (d, 2H, J=8.5 Hz), 6.69 (d, 1H, J=16 Hz), 6.20 (dt, 1H, J=16 Hz,7.5 Hz), 4.56 (d, 2H, J=7 Hz), 3.54 (t, 1H, J=7 Hz), 2.15 (t, 2H, J=7 Hz), 1.65-1.80 (m, 5H), 1.36-1.46 (m, 1H), 1.14-1.30 (m, 3H), 0.90-1.02 (m, 2H).

### C. (E)-{2-Amino-5-(3-cyclohexylprop-1-enyl)-4-[(dicyclohexylamino)methyl]thiophen-3-yl}(4-chlorophenyl)methanone

A stirred, cooled (3° C.) solution of the title B compound (328 mg, 0.63 mmol) and TEA (0.175 mL, 1.2 mmol) in anhydrous DCM (5 mL) is treated under nitrogen with methanesulfonyl chloride (103 mg, 0.90 mmol) in anhydrous DCM (0.5 mL), and stirred at 3° C. for 1 h. More TEA (0.175 mL, 1.2 mmol) and methanesulfonyl chloride (103 mg, 0.90 mmol) are added, and the mixture is stirred for 1 h, warmed to RT, then diluted with DCM (15 mL). The solution is washed with a 1:1-mixture of water and saturated sodium bicarbonate (10 mL), dried (Na₂SO₄), filtered, concentrated in vacuo, and the residue is dissolved in a 2:1-mixture of ACN and THF (9 mL). Dicyclohexylamine (1.0 mL, 5 mmol) is added, and the mixture is heated to 60° C. for 1.5 h, concentrated in vacuo, and dissolved in DCM (25 mL). A 0.1N sodium hydroxide solution (20 mL) is added and the mixture stirred a few minutes, and the layers are allowed to separate. The organic solution is washed with water (2×10 mL), brine (10 mL), dried (Na₂SO₄), filtered, and concentrated in vacuo. The residue is dissolved in a 9:1-mixture of EtOH and water (5 mL), cooled in an ice bath, and treated with ethylenediamine (0.20 mL, 3 mmol) in a 9:1-mixture of EtOH and water (1 mL). The mixture is stirred for 1.5 h at RT, then diluted with DCM (25 mL), dried (Na₂SO₄), filtered, and added to a pad of silica gel. This is eluted with EtOAc and the filtrate is concentrated in vacuo. The residue is dissolved in minimal DCM/heptane and the solution loaded onto a silica gel column which is eluted with a 1:4-mixture of EtOAc and heptane to afford a yellow solid. Trituration with ACN affords (E)-{2-amino-5-(3-cyclohexylprop-1-enyl)-4-[(dicyclohexylamino)methyl]thiophen-3-yl}(4-chlorophenyl)methanone as a yellow solid, m.p.: 147-150° C. MS: 553.2 (M+H). ¹H NMR (CDCl₃): δ 7.63 (d, 2H, J=8.5 Hz), 7.37 (d, 2H, J=8.5 Hz), 6.63 (d, 1H, J=16 Hz), 5.69 (dt, 1H, J=16 Hz,7.5 Hz), 5.60 (br s, 2H), 3.42 (s, 2H), 2.15 (t, 2H, J=7 Hz), 1.85-1.95 (m, 2H), 1.66-1.76 (m, 4H), 1.56-1.63 (m, 4H), 1.45-1.52 (m, 2H).

### EXAMPLE 38

### (E)-{2-amino-5[3-(4-chlorophenyl)prop-1-enyl]-4-[(dicyclohexylamino)methyl]thiophen-3-yl}(4-chlorophenyl)methanone, Method F

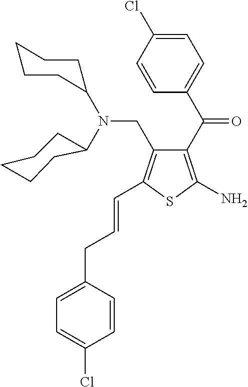

### A. 2-[5-Bromo-3-(4-chlorobenzoyl)-4-(hydroxymethyl)thiophen-2-yl]isoindole-1,3-dione,

A stirred solution of the title A compound of Example 17 (8.09 g, 15 mmol) in THF (250 mL) is treated with 10% aqueous sodium bicarbonate (100 mL) and refluxed for 14 h, then cooled and the layers are separated. The organic layer is concentrated in vacuo and the residue is taken up in toluene (500 mL) and glacial acetic acid (5 mL), refluxed for 19 h, treated with additional glacial acetic acid (5 mL), and refluxed for 5 h more. The mixture is concentrated in vacuo, and the residue is taken up in DCM, loaded onto a pad of silica gel, and eluted with a 1:1-mixture of EtOAc and heptane. The concentrated filtrate is dissolved in ACN, treated with powdered charcoal (3 g), warmed and stirred for a few minutes, and filtered through Celite®. The filtrate is concentrated in vacuo and the residual solid recrystallized from EtOAc/heptane (2 crops) to afford 2-[5-bromo-3-(4-chlorobenzoyl)-4-(hydroxymethyl)thiophene-2-yl]isoindol-1,3-dione as a very pale tan solid, m.p.: 170-172° C. MS: 499.8 (M+Na). ¹H NMR (CDCl₃): δ 7.71-7.78 (m, 4H), 7.59 (d, 2H, J=8.5 Hz), 7.16 (d, 2H, J=8.5 Hz), 4.61 (d, 2H, J=6 Hz).

### B. 3-(4-Chlorophenyl)propyne

An ice-cooled (3° C.) stirred solution of 4-chlorobenzaldehyde (1.41 g, 10 mmol) in anhydrous THF (20 mL) under nitrogen is treated dropwise with 0.5 N ethynyl magnesium bromide (24 mL, 12 mmol), and the mixture is stirred for 30 min at 3° C., for 30 min at RT, and recooled (3° C.). Saturated aqueous ammonia (2.5 mL) is added, followed by water (10 mL), and the mixture is stirred a few minutes, then separated. The aqueous solution is extracted with ether (20 mL) and the combined organic solution is washed with water (25 mL), dried (MgSO₄), filtered, and concentrated in vacuo. The residue is dissolved in minimal heptane/DCM and loaded onto a silica gel column and eluted with a 3:1-mixture of heptane and EtOAc to afford of 3-(4-chlorophenyl)propyn-3-ol, which is used as is without further characterization. The intermediate 3-(4-chlorophenyl)propyn-3-ol is dissolved in anhydrous DCM (20mL) in a 100 mL flask under nitrogen and treated with triethylsilane (3.2 mL, 20 mmol). The stirred mixture is cooled (3° C.), treated with trifluoroacetic acid (3 mL, 40 mmol), stirred at RT for 2 h, and recooled (3° C.). Aqueous sodium bicarbonate is carefully added until trifluoroacetic acid is neutralized, and the layers are separated. The aqueous solution is extracted with DCM (10 mL) and the combined organic solution is dried (Na₂SO₄), filtered, and concentrated in vacuo. The residue is dissolved in heptane and loaded onto a silica gel column and eluted with heptane to afford 3-(4-chlorophenyl)propyne as a colorless oil, ¹H NMR (CDCl₃): δ 7.29 (m, 4H), 3.58 (d, 2H, J=3Hz), 2.20 (t, 1H, J=3Hz).

### C. (E)-2-{3-(4-Chlorobenzoyl)-5-[3-(4-chlorophenyl)prop-1-enyl]-4-(hydroxymethyl)thiophen-2-yl}isoindole-1,3-dione

A stirred, cooled (3° C.) solution of the title B compound (0.377 g, 2.55 mmol) in anhydrous THF (3 mL) under nitrogen is treated dropwise with 1 N catecholborane/THF (3 mL, 3 mmol), and the mixture is refluxed for 2 h and concentrated in vacuo. The residue is cooled on ice and quenched with 1 N HCl (6 mL), then DCM (20 mL) is added and the mixture stirred a few minutes and the layers are separated. The organic solution is washed with water (2×15 mL), dried (Na₂SO₄), filtered, then gently concentrated to 5 mL volume and diluted with 1,4-dioxane (10 mL). The remaining DCM is gently removed in vacuo, and the solution is transferred to a 3-neck 50 mL flask under nitrogen. To the flask are added the title A compound (596 mg, 1.25 mmol) and two drops of water, and the solution is degassed under a stream of nitrogen for 10 min. The mixture is treated with [1,1-bis(diphenylphosphino)-ferrocene]dichloropalladium (II) methylene chloride complex (102 mg, 0.125 mmol) and cesium fluoride (0.60 g, 4 mmol), heated at 45° C. for 45 min with stirring, and at 65° C. for 10 h, then cooled to RT (reaction complete by LCMS analysis). DCM (25 mL) is added, and the mixture is filtered through Celite® (rinse with DCM). The residue is taken up in 3% of acetic acid in toluene (10 mL) and refluxed for 3 h, then cooled to RT and treated with isopropanol (3 mL), water (5 mL), and saturated sodium bicarbonate (5 mL). The layers are separated and the aqueous solution extracted with EtOAc (15 mL). The combined organic solution is dried (MgSO₄), filtered through Celite®, and the filtrate is concentrated in vacuo. The residue is dissolved in minimal DCM and loaded onto a silica gel column, then eluted sequentially with 2%, then 3%, then 4% of EtOAc in DCM to afford (E)-2-{3-(4-chlorobenzoyl)-5-[3-(4-chlorophenyl)prop-1-enyl]-4-(hydroxymethyl)thiophen-2-yl}isoindole-1,3-dione as a light tan foam, MS: 530.0 (M-OH). ¹H NMR (CDCl₃): δ 7.73 (m, 4H), 7.57 (d, 2H, J=8.5 Hz), 7.30 (d, 2H, J=8.5 Hz), 7.15 (m, 4H), 6.78 (d, 1H, J=16 Hz), 6.30 (dt, 1H, J=16 Hz,7.5 Hz), 4.55 (d, 2H, J=7 Hz), 3.55 (m, 3H).

### D. (E)-{2-Amino-5-[3-(4-chlorophenyl)prop-1-enyl]-4-[(dicyclohexylamino)methyl]thiophen-3-yl}(4-chlorophenyl)methanone

A stirred, cooled (3° C.) solution of the title C compound (180 mg, 0.328 mmol) and TEA (0.07 mL, 0.5 mmol) in anhydrous DCM (3 mL) under nitrogen is treated with methanesulfonyl chloride (46 mg, 0.40 mmol) in anhydrous DCM (0.5 mL), and stirred at 3° C. for 1 h. More TEA (0.07 mL, 0.5 mmol) and methanesulfonyl chloride (46 mg, 0.40 mmol) are added, and the mixture is stirred 30 min, then warmed to RT, and diluted with DCM (10 mL). The solution is washed with water (10 mL), dried (Na₂SO₄), filtered, and partially concentrated in vacuo. Dicyclohexylamine (0.4 mL, 2 mmol) and ACN (4 mL) are added, and the remaining DCM is removed. THF (2 mL) is added, and the mixture is heated at 60° C. for 2 h, concentrated in vacuo, and the mixture is taken up in DCM (25 mL). 0.1 N Sodium hydroxide (10 mL) is added and the mixture is stirred a few minutes and the layers are separated. The organic solution is washed with water (2×10 mL), brine (10 mL), dried (Na₂SO₄), filtered, and concentrated in vacuo. The residue is taken up in a 9:1-mixture of EtOH and water (3 mL), cooled in an ice bath, and treated with a solution of ethylenediamine (0.12 mL, 1.8 mmol) in a 9:1-mixture of EtOH and water (1 mL). The mixture is stirred for 2 h at RT, then diluted with DCM (20 mL), dried (Na₂SO₄), filtered, and added to a pad of silica gel. This is eluted with EtOAc and the filtrate is concentrated in vacuo. The residue is dissolved in minimal DCM and the solution is loaded onto a silica gel column which is eluted with 10% of EtOAc in DCM, then with a 3:1-mixture of heptane and EtOAc to afford a yellow solid. Trituration with ACN affords (E)-{2-amino-5[3-(4-chlorophenyl)prop-1-enyl]-4-Rdicyclohexylamino)methyl)thiophen-3-yl}(4-chlorophenyl)methanone as a yellow solid, m.p.: 143-146° C. MS: 581.2 (M+1). ¹H NMR (CDCl₃): δ 7.61 (d, 2H, J=8.5 Hz), 7.38 (d, 2H, J=8.5 Hz), 7.26 (d, 2H, J=8.5 Hz), 7.13 (d, 2H, J=8.5 Hz), 6.77 (d, 1H, J=16 Hz), 5.78 (dt, 1H, J=16 Hz,7.5 Hz), 5.67 (br s, 2H), 3.45 (d, 2H, J=7.5 Hz), 3.38 (s, 2H), 1.94 (m, 2H), 1.57-1.64 (m, 4H), 1.45-1.53 (m, 2H), 1.30-1.38 (m, 4H), 0.80-1.06 (m, 10H).

What is claimed is:

1. A compound of formula (I) 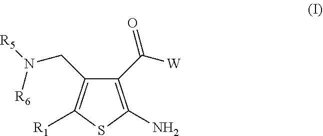
wherein
W is aryl, substituted aryl, heteroaryl, or substituted heteroaryl;
R₁ is hydrogen, alkyl, substituted alkyl, cycloalkyl, substituted cycloalkyl, alkenyl, substituted alkenyl, alkynyl, substituted alkynyl, aryl, substituted aryl, heteroaryl, or substituted heteroaryl;
R₅ and R₆ are, independently from each other, hydrogen, alkyl, substituted alkyl, aryl, substituted aryl, cycloalkyl, substituted cycloalkyl, heterocyclyl, or substituted heterocyclyl;
or a pharmaceutically acceptable salt thereof.

2. A compound according to claim 1 having formula (IA) 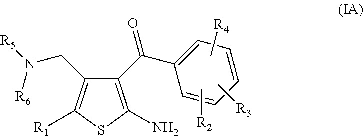
wherein
R₁ is hydrogen, alkyl, substituted alkyl, cycloalkyl, substituted cycloalkyl, alkenyl, substituted alkenyl, alkynyl, substituted alkynyl, aryl, substituted aryl, heteroaryl, or substituted heteroaryl;
R₂, R₃, and R₄ are, independently from each other, hydrogen, alkyl, substituted alkyl, aryl, substituted aryl, cycloalkyl, substituted cycloalkyl, halogen, hydroxyl, nitro, cyano, alkoxy, or substituted alkoxy; or
R₂ and R₃ combined are alkylene which together with the carbon atoms to which they are attached form a 4- to 7-membered fused ring, provided that R₂ and R₃ are attached to carbon atoms adjacent to each other;
R₅ and R₆ are, independently from each other, hydrogen, alkyl, substituted alkyl, aryl, substituted aryl, cycloalkyl, substituted cycloalkyl, heterocyclyl, or substituted heterocyclyl;
or a pharmaceutically acceptable salt thereof.

3. A compound according to claim 2 having formula (IB) 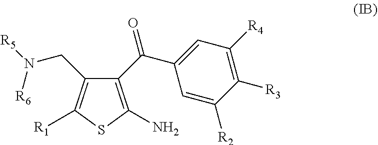
wherein
R₁ is hydrogen, alkyl, substituted alkyl, cycloalkyl, substituted cycloalkyl, alkenyl, substituted alkenyl, alkynyl, substituted alkynyl, aryl, substituted aryl, heteroaryl, or substituted heteroaryl;
R₂, R₃, and R₄ are, independently from each other, hydrogen, alkyl, substituted alkyl, aryl, substituted aryl, cycloalkyl, substituted cycloalkyl, halogen, hydroxyl, nitro, cyano, alkoxy, or substituted alkoxy;
R₅ and R₆ are, independently from each other, hydrogen, alkyl, substituted alkyl, aryl, substituted aryl, cycloalkyl, substituted cycloalkyl, heterocyclyl, or substituted heterocyclyl.
or a pharmaceutically acceptable salt thereof.

4. A compound according to claim 3, wherein
R₁ is hydrogen, alkyl, substituted alkyl, aryl, or substituted aryl;
or a pharmaceutically acceptable salt thereof.

5. A compound according claim 4, wherein
R₁ is monocyclic aryl, or substituted monocyclic aryl;
or a pharmaceutically acceptable salt thereof.

6. A compound according to claim 4, wherein
R₅ and R₆ are, independently from each other, alkyl, or substituted alkyl;
or a pharmaceutically acceptable salt thereof.

7. A compound according to claim 4, wherein
R₂ and R₄ are hydrogen;
or a pharmaceutically acceptable salt thereof.

8. A compound according to claim 7, wherein
R₃ is halogen, cyano, or trifluoromethyl;
or a pharmaceutically acceptable salt thereof.

9. A compound according to claim 4, wherein
R₂ and R₃ are hydrogen;
or a pharmaceutically acceptable salt thereof.

10. A compound according to claim 9, wherein
R₄ is halogen, cyano, or trifluoromethyl;
or a pharmaceutically acceptable salt thereof.

11. A compound according to claim 1 selected from the group consisting of:
{2-Amino-4-[(diisopropylamino)methyl]-5-phenylthiophen-3-yl}(4-chlorophenyl)methanone;
{2-Amino-4-[(dicyclohexylamino)methyl]-5-phenylthiophen-3-yl}(4-chlorophenyl)methanone;
{2-Amino-4-[(diethylamino)methyl]-5-phenylthiophen-3-yl}(4-chlorophenyl)methanone;
{2-Amino-4-[(diallylamino)methyl]-5-phenylthiophen-3-yl}(4-chlorophenyl)methanone;
{2-Amino-4-[(dipropylamino)methyl]-5-phenylthiophen-3-yl}(4-chlorophenyl)methanone;
{2-Amino-4-[(t-butyl(methyl)amino)methyl]-5-phenylthiophen-3-yl}(4-chlorophenyl)methanone;
{2-Amino-4-[(benzyl(methyl)amino)methyl]-5-phenylthiophen-3-yl}(4-chlorophenyl)methanone;
{2-Amino-4-[(dimethylamino)methyl]-5-phenylthiophen-3-yl}(4-chlorophenyl)methanone;
{2-Amino-4-[(butyl(methyl)amino)methyl]-5-phenylthiophen-3-yl}(4-chlorophenyl)methanone;
{2-Amino-5-(4-cyanophenyl)-4-[(dicyclohexylamino)methyl]thiophen-3-yl}(4-chlorophenyl)methanone;
{2-Amino-5-(4-t-butylphenyl)-4-[(dicyclohexylamino)methyl]thiophen-3-yl}(4-chlorophenyl)methanone;
{2-Amino-5-(4-chlorophenyl)-4-[(dicyclohexylamino)methyl]thiophen-3-yl}(4-chlorophenyl)methanone;
{2-Amino-4-[(dicyclohexylamino)methyl]-5-(2-fluorophenyl)thiophen-3-yl}(4-chlorophenyl)methanone;
{2-Amino-4-[(dicyclohexylamino)methyl]-5-(3-fluorophenyl)thiophen-3-yl}(4-chlorophenyl)methanone;
{2-Amino-4-[(dicyclohexylamino)methyl]-5-(4-fluorophenyOthiophen-3-yl}(4-chlorophenyl)methanone;
{2-Amino-4-[(dibutylamino)methyl]-5-phenylthiophen-3-yl}(4-chlorophenyl)methanone;
{2-Amino-4-[(diisobutylamino)methyl]-5-phenylthiophen-3-yl}(4-chlorophenyl)methanone;
{2-Amino-4-[(dipentylamino)methyl]-5-phenylthiophen-3-yl}(4-chlorophenyl)methanone;
{2-Amino-4-[(dicyclopentylamino)methyl]-5-phenylthiophen-3-yl}(4-chlorophenyl)methanone;
{2-Amino-4-[(cyclohexyl(propyl)amino)methyl]-5-phenylthiophen-3-yl}(4-chlorophenyl)methanone;
{4-[(Allyl(cyclohexyDamino)methyl]-2-amino-5-phenylthiophen-3-yl)}(4-chlorophenyl)methanone;
{4-[(2-methoxy-2-oxoethyl)methylamino)methyl]-2-amino-5-phenylthiophen-3-yl)}(4-chloropheny)methanone;
{2-Amino-4-[(bis(2-methoxyethyl)amino)methyl]-5-phenylthiophen-3-yl}(4-chlorophenyl)methanone;
{2-Amino-4-[(dineopentylamino)methyl]-5-phenylthiophen-3-yl}(4-chlorophenyl)methanone;
{2-Amino-4-[(1-adamantylamino)methyl]-5-phenylthiophen-3-yl}(4-chlorophenyl)methanone;
{2-Amino-4-[((3,4-dichlorophenyl)(methyl)amino)methyl]-5-phenylthiophen-3-yl}(4-chlorophenyl)methanone;
{2-Amino-4-[((4-methoxyphenyl)(methyl)amino)methyl]-5-phenylthiophen-3-yl}(4-chlorophenyl)methanone;
{2-Amino-4-[((4-methylphenyl)(methyl)amino)methyl]-5-phenylthiophen-3-yl}(4-chlorophenyl)methanone;
{2-Amino-5-(3,4-dichlorophenyl)-4-[(dicyclohexylamino)methyl]thiophen-3-yl}(4-chlorophenyl)methanone;
{2-Amino-4-[(dicyclohexylamino)methyl]-5-(4-isopropoxyphenyl)thiophen-3-yl}(4-chlorophenyl)methanone;
{2-Amino-4-[(dicyclohexylamino)methyl]-5-(3,5-difluorophenyl)thiophen-3-yl}(4-chlorophenyl)methanone;
{2-Amino-4-[(dicyclohexylamino)methyl]-5-(4-methylphenyl)thiophen-3-yl}(4-chlorophenyl)methanone;
{2-Amino-4-[(dicyclohexylamino)methyl]-5-(4-methoxyphenyl)thiophen-3-yl}(4-chlorophenyl)methanone;
{2-Amino-4-[(diethylamino)methyl]thiophen-3-yl}(4-chlorophenyl)methanone;
{2-Amino-4-[(dipropylamino)methyl]thiophen-3-yl}(4-chlorophenyl)methanone;
{2-Amino-4-[(diisopropylamino)methyl]thiophen-3-yl}(4-chlorophenyl)methanone;
{2-Amino-4-[(methyl(phenyl)amino)methyl]thiophen-3-yl}(4-chlorophenyl)methanone;
{2-Amino-4-[(methyl(phenyl)amino)methyl]thiophen-3-yl}(4-chlorophenyl)methanone;
{2-Amino-4-[(4-fluorophenyl(methyl)amino)methyl]thiophen-3-yl}(4-chlorophenyl)methanone;
{2-Amino-4-[(4-chlorophenyl(methyl)amino)methyl]thiophen-3-yl}(4-chlorophenyl)methanone;
{2-Amino-4-[(methyl(4-trifluoromethylphenyl)amino)methyl]thiophen-3-yl}(4-chlorophenyl)methanone;
{2-Amino-4-[(benzyl(methyl)amino)methyl]-5-methylthiophen-3-yl}(4-chlorophenyl)methanone;
{2-Amino-[(dicyclohexylamino)methyl]-5-(furan-3-yl)thiophen-3-yl}(4-chlorophenyl)methanone;
{2-Amino-4-[(dicyclohexylamino)methyl]-5-(thiophen-2-yl)thiophen-3-yl}(4-chlorophenyl)methanone;
{2-Amino-4-[(dicyclohexylamino)methyl]-5-(furan-2-ypthiophen-3-yl}(4-chlorophenyl)methanone;
{5-Amino-5′-chloro-3-[(dicyclohexylamino)methyl]-2,2′-bithiophen-4-yl}(4-chlorophenyl)methanone;
(E)-{2-Amino-5-(3-cyclohexylprop-1-enyl)-4-[(dicyclohexylamino)methyl]thiophen-3-yl}(4-chlorophenyl)methanone;
(E)-{2-Amino-5-[3-(4-chlorophenyl)prop-1-enyl]-4-[(dicyclohexylamino)methyl]thiophen-3-yl}(4-chlorophenyl)methanone;
{2-Amino-4-[(dicyclohexylamino)methyl]-5-[4-(2-methoxyethoxy)phenyl]thiophen-3-yl}(4-chlorophenyl)methanone;
{2-amino-4-[(dicyclohexylamino)methyl]-5-(pyridin-2-yl)thiophen-3-yl}(4-chlorophenyl)methanone;
{2-amino-4-[(dicyclohexylamino)methyl]-5-(pyridin-3-yl)thiophen-3-yl}(4-chlorophenyl)methanone; and
{2-amino-4-[(dicyclohexylamino)methyl]-5-(pyridin-4-yl)thiophen-3-yl}(4-chlorophenyl)methanone;
or a pharmaceutically acceptable salt thereof.

12. A pharmaceutical composition comprising a therapeutically effective amount of a compound of claim 1 in combination with one or more pharmaceutically acceptable carriers.

13. A pharmaceutical composition according to claim 12 for the treatment of pain, cardiac disease or disorder, neurological disease or injury, sleep disorder, epilepsy and depression.

14. A pharmaceutical composition according to claim 13, wherein the pain is neuropathic pain.

15. A pharmaceutical composition according to claim 13, wherein the pain is inflammatory pain.

16. A pharmaceutical composition according to claim 13, wherein the cardiac disease or disorder is selected from the group consisting of cardiac disarrhythmias, angina, myocardial infarction and stroke.

17. A method for the modulation of the A₁ adenosine receptor in mammals which method comprises administering to a mammal in need thereof a therapeutically effective amount of a compound of formula (I) 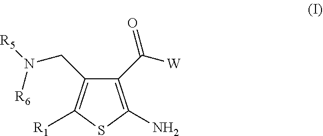
wherein
W is aryl, substituted aryl, heteroaryl, or substituted heteroaryl;
R₁ is hydrogen, alkyl, substituted alkyl, cycloalkyl, substituted cycloalkyl, alkenyl, substituted alkenyl, alkynyl, substituted alkynyl, aryl, substituted aryl, heteroaryl, or substituted heteroaryl;
R₅ and R₆ are, independently from each other, hydrogen, alkyl, substituted alkyl, aryl, substituted aryl, cycloalkyl, substituted cycloalkyl, heterocyclyl, or substituted heterocyclyl;
or a pharmaceutically acceptable salt thereof.

18. A method for the treatment of conditions mediated by the A₁ adenosine receptor in mammals which method comprises administering to a mammal, in need thereof, a therapeutically effective amount of a compound of formula (I) 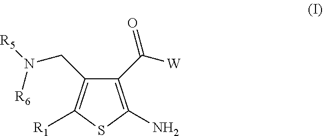
wherein
W is aryl, substituted aryl, heteroaryl, or substituted heteroaryl;
R₁ is hydrogen, alkyl, substituted alkyl, cycloalkyl, substituted cycloalkyl, alkenyl, substituted alkenyl, alkynyl, substituted alkynyl, aryl, substituted aryl, heteroaryl, or substituted heteroaryl;
R₅ and R₆ are, independently from each other, hydrogen, alkyl, substituted alkyl, aryl, substituted aryl, cycloalkyl, substituted cycloalkyl, heterocyclyl, or substituted heterocyclyl;
or a pharmaceutically acceptable salt thereof.

19. A method according to claim 18, wherein the condition mediated by the A₁ adenosine receptor is selected from the group consisting of pain, a cardiac disease or disorder, neurological disease or injury, sleep disorders, epilepsy and depression.

20. A method according to claim 19, wherein the pain is neuropathic pain.

21. A method according to claim 19, wherein the pain is inflammatory pain.

22. A method according to claim 19, wherein the cardiac disease or disorder is selected from the group consisting of cardiac disarrhythmias, angina, myocardial infarction and stroke.
